(19) United States
(12) Patent Application Publication
(10) Pub. No.: US 2026/0224363 A1
(43) Pub. Date: Aug. 6, 2026

(54) SYSTEMS AND METHODS FOR IMPROVED DEPLOYMENT OF PROSTHETIC VALVES

(71) Applicant: Edwards Lifesciences Corporation, Irvine, CA (US)

(72) Inventors: Mohammad Reza Salari, Irvine, CA (US); Yevgeniy Davidovich Kaufman, Denver, CO (US)

(21) Appl. No.: 19/636,854

(22) Filed: Apr. 1, 2026

Related U.S. Application Data
(63) Continuation of application No. PCT/US2024/049418, filed on Oct. 1, 2024.
(60) Provisional application No. 63/542,259, filed on Oct. 3, 2023.

(51) Int. Cl. A61F 2/24

(52) U.S. Cl. CPC A61F 2/2436

(57) ABSTRACT

Apparatuses, systems, and methods for various devices, such as prosthetic valves. Some examples are directed to improvements in anchoring at a native heart valve, which may reduce possible trauma to a treatment or implantation site. Compliant and flexible anchoring features and methods are disclosed to reduce force upon a treatment or implantation site including heart tissue that surrounds leaflets of a mitral or tricuspid valve. Improved mechanisms for deployment of devices including motorized methods are also disclosed.

### CROSS-REFERENCE TO RELATED APPLICATIONS

This application is a continuation of International Application No. PCT/US2024/049418, filed October 1, 2024, which designates the United States and was published in English by the International Bureau on April 10, 2025 as WO 2025/075966, and which claims priority to U.S. Provisional Application No. 63/542,259, filed October 3, 2023.

The entire contents of each of the above documents are incorporated by reference herein.

### BACKGROUND

### Field

Features of the disclosure relate to implants, including prosthetic valves for replacing the function of a native heart valve.

### Background

Human heart valves, which include the aortic, pulmonary, mitral, and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves’ ability to control blood flow. Such impairments reduce the heart’s blood-pumping efficiency and can be a debilitating and life-threatening condition. For example, valve deficiencies can lead to conditions such as heart hypertrophy and dilation of the ventricle. Thus, extensive efforts have been made to develop methods and apparatuses to repair or replace impaired heart valves.

Substantial effort has been dedicated to developing replacement heart valves, particularly tissue-based replacement heart valves that can be delivered with less trauma to the patient than through open heart surgery. Proper deployment and sealing of a replacement valve to an implantation site is desirable.

### SUMMARY

This summary is meant to provide some examples and is not intended to be limiting of the scope of the disclosure in any way. For example, any feature included in an example of this summary is not required by the claims, unless the claims explicitly recite the features. Also, the features, components, steps, concepts, etc. described in some implementations in this summary and elsewhere in this disclosure can be combined in a variety of ways. Various features and steps as described elsewhere in this disclosure may be included in the examples summarized here.

In some implementations herein, implantable protheses or implantable devices, such as replacement heart valves and/or valve repair devices, are provided. In some implementations, prostheses or devices herein include compliant anchoring mechanisms for prosthetic heart valves (aka replacement heart valves). In some implementations, improved delivery systems for better controlling rotational and/or axial movement of a prosthesis or device (e.g., a prosthetic heart valve, a repair device, etc.) are provided. In some implementations, improved imaging systems for enhanced visualization during the implantation procedure are provided. These and other concepts may be utilized alone or in combination with other concepts described herein to facilitate the implantation of a prosthetic heart valve.

In some implementations, prostheses or devices (e.g., heart valves, repair devices, replacement devices, etc.) with improved anchoring are disclosed. In some implementations, the prostheses or devices may be configured for use and/or implantation at a treatment site (e.g., an implantation site, a repair site, etc.). In some implementations, the treatment site can be a native mitral valve or a native tricuspid valve (although other valves, such as aortic, pulmonic, eustachian, etc., for implantation are contemplated).

In some implementations, the improved anchoring features and/or methods are configured to improve stability while reducing trauma at or around the treatment or implantation site. For example, the systems, devices, methods, etc. herein are configured to reduce the possibility of impact, pressure, and/or potential bruising (e.g., pseudo-aneurysm, myocardium puncture, or electrical conduction disturbances).

In some implementations, improved anchoring systems disclosed herein comprise compliant anchoring structures. In some implementations, the anchors are compliant or flexible anchors and are configured to reduce trauma to surrounding tissue at a treatment or implantation site.

In some implementations, mechanisms and/or methods for operation of a delivery system are also disclosed herein. In some implementations, the mechanisms/methods are provided for improved operation and deployment of a device or implant from an elongate delivery catheter, thereby allowing treatment (e.g., repair, implantation, etc.) via a percutaneous, non-invasive procedure. Various other improvements are disclosed.

In some implementations, a prosthesis or device (e.g., prosthetic heart valve, repair device, replacement device, implant, treatment device, etc.) for use and/or deployment to a native heart valve is provided. In some implementations, the device (e.g., the prosthetic heart valve, etc.) includes a support structure having an inlet end portion. In some implementations, the support structure includes an outlet end portion. The prosthesis or device can include any of the features of other prostheses or devices described herein, and can be used with any of the delivery systems described herein.

In some implementations, the support structure includes a passageway extending therethrough.

In some implementations, the support structure may include an inner frame having an inlet end portion and an outlet end portion. In some implementations, the support structure may include an outer frame including an inlet end portion.

In some implementations, the inlet end portion of the outer frame is coupled to the inlet end portion of the inner frame.

In some implementations, the outer frame is formed with a plateau portion or flange that extends radially outward from the inlet end portion of the outer frame to form a shoulder. In some implementations, an axially extending portion extends axially from the shoulder toward an outlet end portion.

In some implementations, the shoulder angles the plateau portion relative to the axially extending portion. In some implementations, the plateau portion includes a flexibility feature that allows the plateau portion to deflect for axial translation of the inner frame relative to the shoulder.

In some implementations, a valve portion is positioned within the passageway of the support structure. In some implementations, the valve portion comprises a plurality of prosthetic valve leaflets configure to allow blood to flow through the passageway in one direction and inhibit blood flow in an opposite direction.

In some implementations, the device (e.g., prosthetic heart valve, treatment device, etc.) includes a support structure having an inlet end portion and an outlet end portion. In some implementations, the support structure comprises a passageway.

In some implementations, a valve portion is positioned within the passageway of the support structure. In some implementations, the valve portion comprises a plurality of prosthetic valve leaflets. In some implementations, the valve portion permits flow of blood through the passageway in one direction for replacing the function of the native heart valve.

In some implementations, one or more anchors extend radially outward from the support structure.

In some implementations, the one or more anchors each have a hook shape.

In some implementations, the one or more anchors each include an anchor arm that is split or forked along the end.

In some implementations, the one or more anchors each include a tip portion with an undulation or serpentine configuration for increasing compliance and flexibility along the tip portion, which can help to reduce trauma to surrounding tissue.

In some implementations, the one or more anchors include a tip portion having a compliant braid.

In some implementations, the one or more anchors include a tip portion forming a loop.

In some implementations, the one or more anchors include more than one or multiple of the above features.

In some implementations, the device comprises a sealing body.

In some implementations, the device comprises one or more flexibility features.

In some implementations, a delivery system for advancing a device (e.g., a prosthetic heart valve, a repair device, a replacement device, a treatment device, etc.) through a subject’s vasculature is provided. In some implementations, the delivery system is operable and/or is configured to use, operate, and/or implant the device at, proximate, or within a native heart valve. The delivery system can be used with any of the devices, prostheses, systems, etc. described above or elsewhere herein.

In some implementations, the delivery system comprises a catheter shaft for advancement of the device to a treatment site and/or implantation site. In some implementations, the catheter shaft includes a proximal end portion and a distal end portion.

In some implementations, at least a portion of the catheter shaft includes a retention area for the device. In some implementations, the retention area may take the form of a sheath or capsule sized to hold the device (e.g., a prosthetic heart valve, etc.) in a compressed state.

In some implementations, the delivery system may include an actuation mechanism for rotating the device (e.g., prosthetic heart valve, treatment device, etc.) about or relative to an axis of the catheter shaft (e.g., an axis of a distal portion or distal end of the catheter shaft, etc.).

In some implementations, a mechanism for rotating the device provides the clinician with the ability to orient the device (e.g., prosthetic valve, treatment device, etc.) within the native valve in a more desirable manner (e.g., an optimal manner for a particular treatment, a better manner for avoiding damage to sensitive tissue, etc.). This may be particularly advantageous when using a non-cylindrical or asymmetric device, such as for conforming to the shape of a native valve.

In some implementations, the delivery system includes a catheter shaft for advancement of the device (e.g., prosthetic heart valve, treatment device, etc.) to a treatment site (e.g., to an implantation site, a repair site, a replacement site, etc.). In some implementations, the catheter shaft extends (or can extend) along an axis. In some implementations, the catheter shaft includes a proximal end portion and a distal end portion.

In some implementations, at least a portion of the catheter shaft includes a retention area for the device (e.g., for a prosthetic heart valve, etc.).

In some implementations, the delivery system comprises a motor for controlling axial movement of the device along or relative to at least a portion of the catheter shaft. In some implementations, the motor may be positioned along the distal end portion of the delivery system.

In some implementations, the delivery system (e.g., any of the delivery systems described anywhere herein) includes an imaging system.

In some implementations, the catheter shaft includes a nose body. In some implementations, the catheter shaft, or another portion of the delivery system, includes a sheath having a distal end portion attached to the nose body.

In some implementations, the sheath includes an interior lumen for a guide wire to pass through. In some implementations, a portion of the catheter shaft includes a retention area for the device (e.g., prosthetic heart valve, etc.).

In some implementations, the delivery system comprises an imaging system coupled to one or more of the nose body or the sheath. In some implementations, the imaging system is adapted to image an interior portion of a subject’s body from inside of the subject. In some implementations, the imaging system is adapted to image vasculature of a subject’s body from interior of the subject’s vasculature. The subject for any of the devices, systems, or methods herein can be a living subject or a simulation.

In some implementations, a guide wire system (useable with any of the delivery systems and/or devices herein) comprises a guide wire body having a proximal end portion and a distal end portion. In some implementations, an imaging system is coupled to the guide wire body. In some implementations, the imaging system is adapted to image an interior portion of a subject’s body from inside of the subject. In some implementations, the imaging system is adapted to image vasculature of a subject’s body from interior of the subject’s vasculature. Integrating an imaging system into the guide wire has numerous advantages and is applicable to numerous procedures in addition to prosthetic heart valve implantation.

The delivery system can include any of the features of delivery systems described below as well.

Any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1 illustrates a perspective view of a prosthetic valve from an inflow side of the prosthetic valve.

FIG. 2 illustrates a perspective view of a frame of the prosthetic valve shown in FIG. 1.

FIG. 3 illustrates a perspective view of the inner frame shown in FIG. 2.

FIG. 4 illustrates a perspective view of the outer frame shown in FIG. 2.

FIG. 5 illustrates a cross-sectional schematic view of the prosthetic valve shown in FIG. 1.

FIG. 6 illustrates a schematic view of a delivery system approaching a treatment or implantation site.

FIG. 7 illustrates a cross-sectional schematic view of a prosthetic valve within a capsule of a delivery system.

FIG. 8 illustrates a cross-sectional schematic view of the prosthetic valve of FIG. 7 deployed to a treatment or implantation site.

FIG. 9 illustrates a representation of a frame deployed to a treatment or implantation site in a side view.

FIG. 10 illustrates a perspective view of an outer frame.

FIG. 11 illustrates a close-up perspective view of a portion of the outer frame shown in FIG. 10.

FIG. 12 illustrates a plan view of a portion of the outer frame shown in FIG. 10.

FIG. 13 illustrates a representation of a frame deployed to a treatment or implantation site in a side view.

FIG. 14 illustrates a perspective view of an anchor.

FIG. 15 illustrates a top view of a frame including a plurality of anchors having prongs.

FIG. 16 illustrates a schematic view of an anchor having prongs.

FIG. 17 illustrates a side view of a frame including anchors having prongs.

FIG. 18 illustrates a perspective view of an anchor.

FIG. 19 illustrates a top view of a frame including anchors having prongs.

FIG. 20 illustrates a plan view of an anchor including a flexible tip portion.

FIG. 21 illustrates a perspective view of the flexible tip portion shown in FIG. 20.

FIG. 22 illustrates a side cross-sectional view of an anchor.

FIG. 23 illustrates a perspective view of an anchor.

FIG. 24 illustrates a schematic view of an assembly of an anchor having a compliant braid.

FIG. 25 illustrates a side cross-sectional view of an anchor having a compliant braid.

FIG. 26 illustrates a side view of an anchor having a compliant braid.

FIG. 27 illustrates a side view of an anchor having a compliant braid.

FIG. 28 illustrates a side cross-sectional view of an anchor having a compliant braid.

FIG. 29 illustrates a side view of an anchor having a compliant braid.

FIG. 30 illustrates a rear view of the anchor having the compliant braid shown in FIG. 29.

FIG. 31 illustrates a side view of the anchor shown in FIG. 29.

FIG. 32 illustrates a perspective view of an anchor having a loop.

FIG. 33A illustrates a cross-sectional view along line I-I in FIG. 32.

FIG. 33B illustrates a side view of a braid upon a core.

FIG. 34 illustrates a side cross-sectional view of an arm of a loop shown in FIG. 32.

FIG. 35 illustrates a perspective view of a prosthetic heart valve including loops as shown in FIG. 32.

FIG. 36 illustrates a side view of a deflection of a loop as shown in FIG. 32.

FIG. 37 illustrates a top view of a deflection of a loop as shown in FIG. 32.

FIG. 38 illustrates a perspective view of an anchor having a loop and an indicator.

FIGS. 39A and 39B illustrate side views of the anchor shown in FIG. 38.

FIG. 40 illustrates a perspective view of an anchor having a loop and an indicator.

FIGS. 41A and 41B illustrate side views of the anchor shown in FIG. 40.

FIG. 42 illustrates a cross-sectional view of an arm of a loop.

FIG. 43 illustrates a perspective view of an anchor having multiple loops.

FIG. 44 illustrates a side view of the multiple loops shown in FIG. 43.

FIG. 45 illustrates a perspective view of an anchor having a loop.

FIG. 46 illustrates a perspective view of an anchor having a loop and an indicator.

FIGS. 47 and 48 illustrate side views of the anchor shown in FIG. 46.

FIG. 49 illustrates a perspective view of a delivery system.

FIG. 50 illustrates a side schematic view of a distal end portion of a catheter shaft.

FIG. 51 illustrates a detail cross-sectional schematic view of a tip of the catheter shaft.

FIG. 52 illustrates a side cross-sectional schematic view of a guide wire sheath.

FIG. 53 illustrates a side cross-sectional schematic view of a capsule sheath.

FIG. 54 illustrates a side schematic view of a delivery system approaching a treatment or implantation site.

FIG. 55 illustrates a side schematic view of a delivery system deploying an implant to a treatment or implantation site.

FIG. 56 illustrates a side view of a guide wire.

FIG. 57 illustrates a perspective cross-sectional view of a portion of the guide wire shown in FIG. 56.

FIG. 58 illustrates a side schematic view of a delivery system deploying an implant to a treatment or implantation site.

FIG. 59 illustrates a perspective view of an actuation mechanism of a delivery system.

FIG. 60 illustrates a perspective view of an actuation mechanism of a delivery system.

FIG. 61 illustrates a perspective view of an actuation mechanism of a delivery system.

FIG. 62 illustrates a perspective view of an actuation mechanism of a delivery system.

FIG. 63 illustrates a top schematic view of anchors of an implant relative to leaflets of a native heart valve.

FIG. 64 illustrates a top schematic view of anchors of an implant relative to leaflets of a native heart valve.

FIG. 65 illustrates a side cross-sectional schematic view of an actuation mechanism of a delivery system.

FIG. 66 illustrates a side cross-sectional schematic view of an actuation mechanism of a delivery system.

FIG. 67 illustrates a side cross-sectional schematic view of an actuation mechanism of a delivery system.

FIG. 68 illustrates a side cross-sectional schematic view of an actuation mechanism of a delivery system.

FIG. 69 illustrates a side cross-sectional schematic view of a delivery system varying a depth of an implant.

FIG. 70 illustrates a side view of a delivery system including a guide wire.

FIG. 71 illustrates a side cross-sectional schematic view of a guide wire within a lumen of a delivery system.

FIG. 72 illustrates a side cross-sectional schematic view of a guide wire within a lumen of a delivery system.

FIG. 73 illustrates a side cross-sectional schematic view of a guide wire within a lumen of a delivery system.

FIG. 74 illustrates a side cross-sectional schematic view of a guide wire within a lumen of a delivery system.

### DETAILED DESCRIPTION

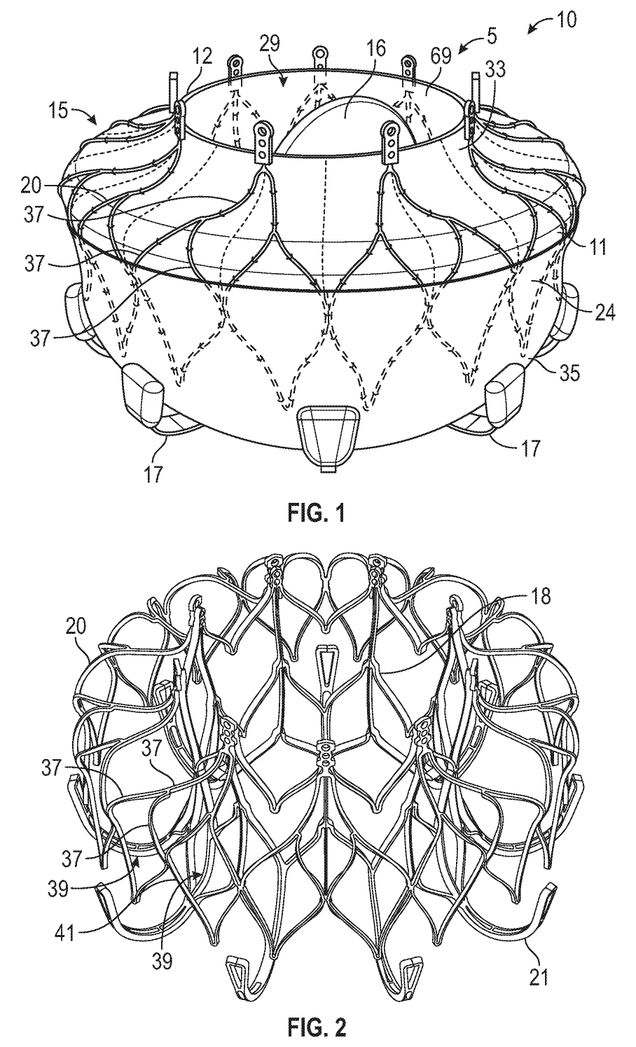

FIG. 1 illustrates a perspective view of a prosthesis or device configured as a replacement device or prosthetic valve 10. While replacement devices or prosthetic valves are used for illustrative purposes in many of the examples herein, it should be understood that the same concepts can be applied to other types of devices, such as treatment devices, repair devices, various implant devices, etc.

In some implementations, the devices herein, e.g., prosthetic valve 10, etc. are configured for use and/or deployment within a subject’s body. The prosthetic valve 10, for example, is deployable to an annulus of a native valve, which may comprise a native mitral valve or a native tricuspid valve. In some implementations, other implantation locations may be utilized such as within an aortic or pulmonary valve, or in other valves or locations within a subject’s body as desired.

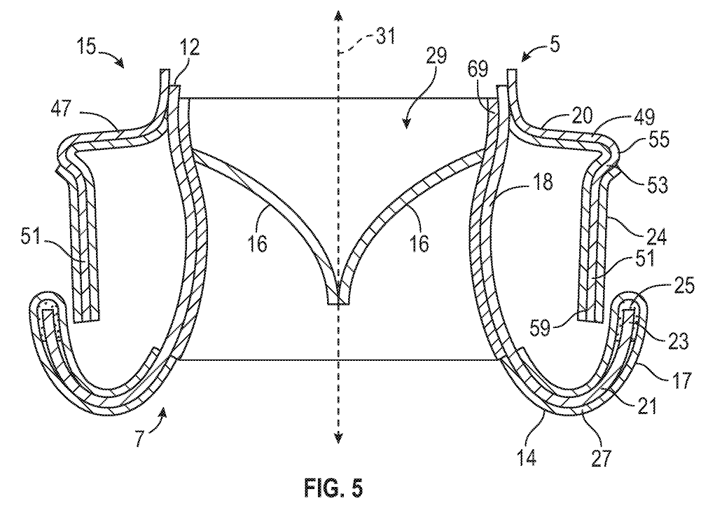

In some implementations, the device or prosthetic valve 10 includes a proximal end 12 or inlet end portion and a distal end 14 or outlet end portion (marked in FIG. 5), and a length therebetween. In some implementations, the device or prosthetic valve 10 further includes a valve portion, preferably formed by a plurality of prosthetic valve leaflets 16.

In some implementations, the valve portion is positioned in a flow channel or passageway for controlling flow through the device or prosthetic valve 10. In some implementations, the flow channel or passageway is formed by a support structure 15 of the device/valve 10. In some implementations, the support structure 15 has a proximal end portion or inlet end portion 5 and a distal end portion or outlet end portion 7 (marked in FIG. 5).

In some implementations, the prosthetic valve leaflets 16 move between opened and closed states to mimic and replace the operation of native valve leaflets. In some implementations, the valve portion is positioned within the passageway of the support structure 15 for permitting flow of blood through the passageway in one direction, thereby replacing the function of a native heart valve.

In some implementations, the prosthetic valve leaflets 16 are made of pericardium, such as bovine or porcine pericardium, or another material as desired. In some implementations, the leaflets are formed of a synthetic (e.g., polymer) material or the valve portion may be a mechanical one-way valve.

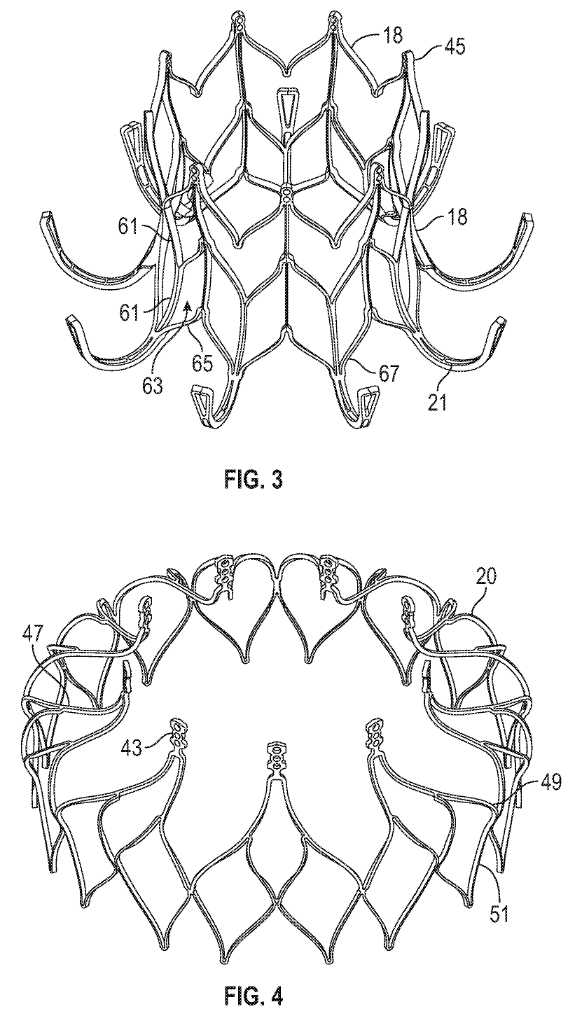

In some implementations, the support structure 15 or valve body surrounds and supports the prosthetic valve leaflets 16. In some implementations, the support structure 15 includes a stent or a frame or a support frame (e.g., a valve frame or inner support stent or inner frame 18 (shown in isolation in FIG. 3) and an outer support stent or outer frame 20 (shown in isolation in FIG. 4), among other forms of frames). In some implementations, the outer support stent or outer frame 20 is part of a sealing body 11 and is spaced from the inner frame 18. In some implementations, the outer frame 20 surrounds the inner frame 18.

In some implementations, the device or prosthetic valve 10 includes one or more anchors 17 for stabilizing the device or prosthetic valve in the body after implantation. The one or more anchors can be used on a variety of types of devices and are not limited to prosthetic valves.

In some implementations, the anchors 17 may particularly anchor to the native valve leaflets of the subject’s heart. In some implementations, the anchors 17 extend around the native valve leaflets to anchor to the native valve leaflets. In some implementations, the anchors 17 comprise distal anchors positioned at the distal end 14 or outlet end portion of the valve 10, or in some implementations are positioned at another position as desired.

In some implementations, the anchors 17 extend or can extend radially outward from the support structure 15. In some implementations, when the device or prosthetic valve is used, deployed, or implanted, the native leaflets are captured between the anchors 17 and the outer frame 20.

In some implementations, each anchor 17 is constructed as a protruding anchor arm extending distally and then curving in a proximal direction to the tip of the respective one of the anchors 17. Such a configuration allows the anchor 17 to extend around a native leaflet and around the distal tip of the leaflet, to hook over the distal tip of the native valve leaflet and be positioned radially outward of an outward facing surface of a leaflet of the native valve.

In some implementations, the anchors 17 are in a hooked configuration as shown in FIGS. 1‒3 and 5 for example. In some implementations, one or more, some, or all of the anchors 17 have a hook shape. The anchors 17 thus resist a force applied in the atrial or proximal direction to the valve 10 and anchor the valve 10 within the native valve annulus.

In some implementations, the anchors 17 are coupled to a distal end portion 67 or outlet end portion of the inner frame 18. Other coupling locations are possible.

In some implementations, the anchors 17 each include a strut arm 21 (marked in FIGS. 2, 3, and 5) that may be covered with covering material. In some implementations, the covering material may comprise one or more layers of padding or cushioning material that cover the strut arm 21. Referring to the cross-sectional view of FIG. 5, for example, the strut arm 21 can include a tip 23 that may be covered with a pad 25. In some implementations, a sleeve 27 may extend along the length of the strut arm 21 and may cover the tip 23 and the pad 25. The sleeve 27 and pad 25 serve to provide a cushion for the anchor 17. The strut arm 21, for example, may be relatively rigid compared to the padding or cushioning material.

Other configurations of anchors 17 may be utilized in some implementations as desired.

In some implementations, prosthetic valve leaflets 16 are positioned within a passageway or flow channel 29 as marked in FIGS. 1 and 5 and move between open and closed states to control flow through the passageway or flow channel 29. As shown in FIG. 5, the proximal end 12 of the prosthetic valve 10 comprises an inflow end of the prosthetic valve 10, and the distal end 14 of the prosthetic valve 10 comprises an outflow end, although other configurations are utilized as desired.

In some implementations, the prosthetic valve leaflets 16 can be positioned around a central axis 31 of the prosthetic valve 10. In some implementations, the inner frame 18 and outer frame 20 each surround the central axis 31 of the prosthetic valve 10.

As illustrated in FIG. 5, when the prosthetic valve leaflets 16 are in the closed position, the passageway is blocked for preventing blood from flowing backwards through the passageway (i.e., blood cannot flow from the outflow end back toward the inflow end).

Referring again to FIG. 1, in some implementations, the device (e.g., prosthetic valve 10, etc.) can include a sealing body 11. In some implementations, the sealing body 11 is positioned radially outward from the support structure (e.g., from an inner frame and/or an outer frame). In some implementations, the sealing body 11 is positioned in a central location. In some implementations, the sealing body seals against a portion of the native valve.

In some implementations, the sealing body is positioned radially outward from prosthetic valve leaflets 16. In some implementations, the sealing body seals against a portion of the native valve.

In some implementations, the sealing body 11 may comprise the outer surface of the valve 10. In some implementations, the sealing body 11 may define the outer diameter of the device and may comprise the outer periphery of the device (e.g., valve 10, etc.). In some implementations, the sealing body 11 includes a proximal portion or inlet end portion having a proximal end 33 and includes a distal portion or outlet end portion having a distal end 35.

In some implementations, the sealing body 11 includes the frame or outer support stent or outer frame 20 and a sealing skirt 24, or in some implementations comprises only a frame or only a sealing skirt as desired.

In some implementations, the outer frame 20 is positioned radially outward from the inner support stent or inner frame 18. In some implementations, the sealing skirt 24 is coupled to the outer frame 20 and comprises the outer portion of the sealing body 11.

In some implementations, the sealing skirt 24 is made of a material that resists fluid flow therethrough, such as a cloth material, woven material, or other material such as a polymer or other material that resists fluid flow therethrough. The material may comprise a fabric. A variety of materials may be utilized for the skirt 24 as desired.

In some implementations, the sealing body 11 abuts a portion of the subject’s heart to reduce fluid flow. In some implementations, the skirt 24 seals a portion of the native valve annulus. For example, the sealing body 11 abuts a surface of a subject’s native valve leaflet to reduce fluid flow between the sealing body 11 and the native leaflet. In some implementations, the sealing body 11 abuts other portions of the subject’s heart to reduce fluid flow as desired.

In some implementations, the sealing body 11 is flexible to allow for movement and conformability to a native valve annulus.

In some implementations, the outer frame 20 is positioned radially outward from the inner frame 18. In some implementations, the outer frame 20 is made from a plurality of struts 37 that are shaped to form a desired shape or profile for the outer frame 20. In some implementations, the plurality of struts 37 form a lattice structure with respective openings 39 (marked in FIG. 2) between the struts 37.

In some implementations, the struts 37 form cells that bound the openings 39. In some implementations, the cells (e.g., cell 41 marked in FIG. 2) comprise closed cells as shown in FIG. 2 with openings that are fully bounded by the struts or comprise opened cells in some implementations with openings that are not fully bounded by struts.

In some implementations, the lattice structure of the outer frame 20 allows the outer frame 20 to move between a collapsed, compressed, or crimped configuration radially outward to an expanded or deployed configuration. In some implementations, the lattice structure is elongated in the collapsed, compressed, or crimped configuration and may shorten in length and expand radially outward in the expanded or deployed configuration.

FIG. 4 illustrates the example outer frame 20 in isolation. In some implementations, the outer frame 20 includes a proximal end portion 43 or inlet end portion. In some implementations, the proximal end portion 43 or inlet end portion couples to a proximal end portion 45 or inlet end portion of the inner frame 18 (marked in FIG. 3). In some implementations, the proximal end portion 43 comprises a coupler in some implementations, for coupling to the proximal end portion 45 of the inner frame. In some implementations, the coupler comprises an eyelet for sutures for coupling to the inner frame 18 or has another configuration in some implementations. Other forms of couplers are utilized in some implementations.

In some implementations, the outer frame 20 includes a plateau portion 47 that extends radially outward from the proximal end portion 43 or inlet end portion of the outer frame 20. The plateau portion 47 may be flat or may have a curvature. In some implementations, the plateau portion 47 extends radially outward to a shoulder 49. In some implementations, the shoulder 49 angles the plateau portion 47 relative to an axially extending portion 51 of the outer frame 20.

Referring to the cross-sectional view of FIG. 5, the shoulder 49 comprises a radially outward bowing portion of the outer frame 20 having a convex curvature. A distal portion 53 of the shoulder 49 extends radially inward such than an apex 55 of the shoulder 49 protrudes radially outward. In some implementations, the axially extending portion 51 extends axially from the distal portion 53 of the shoulder 49 to a distal end portion 59 or outlet end portion of the outer frame 20.

In some implementations, the shoulder 49 resists distal or ventricular movement of the prosthetic valve 10 upon implantation. In some implementations, the shoulder 49, for example, abuts an atrial side of the heart valve annulus to resist the distal or ventricular movement. In some implementations, the axially extending portion 51 extends along and bounds inward facing surfaces of the native valve leaflets.

Other configurations of outer frames may be utilized in some implementations.

Referring to FIG. 3, the inner frame 18 is shown in isolation. In some implementations, the inner frame 18 includes a plurality of struts 61 forming a lattice structure in a similar manner as the outer frame 20. In some implementations, the struts 61, for example, bound openings 63. In some implementations, the struts 61 form closed cells 65 about the openings 63 or form opened cells in some implementations. In some implementations, the lattice structure allows the inner frame 18 to move between a collapsed, compressed, or crimped configuration radially outward to an expanded or deployed configuration in a similar manner as the outer frame 20. In some implementations, the lattice structure is elongated in the collapsed, compressed, or crimped configuration and shortens in length and expands radially outward in the expanded or deployed configuration.

In some implementations, the inner frame 18 includes the proximal end portion 45 or inlet end portion of the inner frame 18 and extends axially to a distal end portion 67 or outlet end portion of the inner frame 18. In some implementations, the strut arms 21 are integral with the struts of the inner frame 18 at the distal end portion 67 and extend radially outward from the distal end portion 67 of the inner frame 18.

In some implementations, a skirt 69 (marked in FIGS. 1 and 5) extends along the interior surface of the inner frame 18 and couples to the prosthetic valve leaflets 16. Sutures or other forms of couplers are utilized to couple the prosthetic valve leaflets 16 to the skirt 69. Other configurations of prosthetic valves are utilized in some implementations.

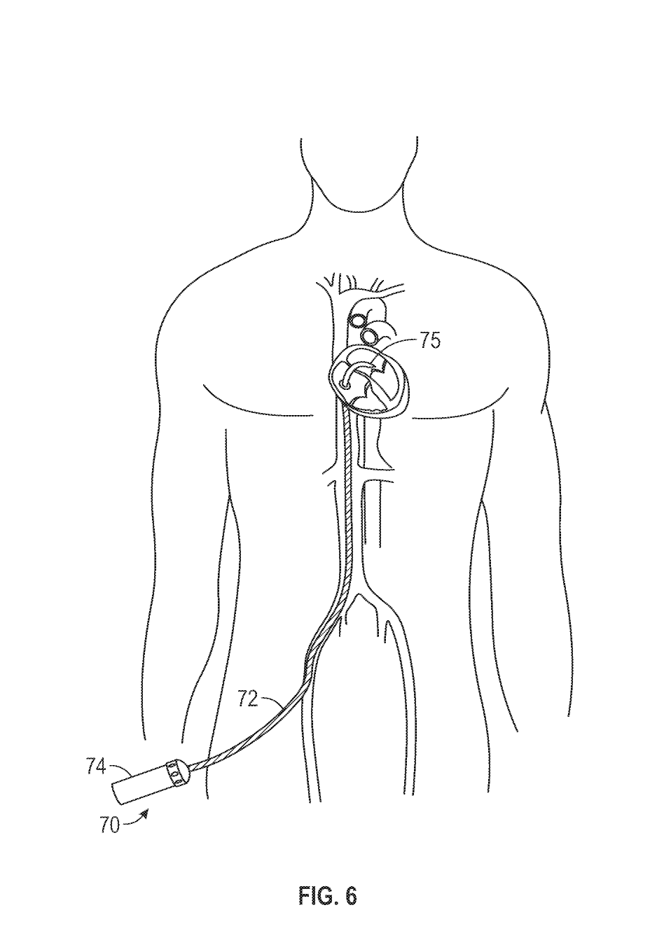

FIG. 6 illustrates one manner of advancing a delivery system 70 or delivery catheter toward a treatment or implantation site. In some implementations, the delivery system 70 includes an elongate shaft or catheter shaft 72 having a proximal portion and a distal portion, with the proximal portion coupled to a housing in the form of a handle 74. In some implementations, the delivery system 70 may be advanced through the vasculature of a subject, which may include the femoral vein as shown in FIG. 6. Other entries may be utilized in some implementations, including transapical, transatrial, transjugular, transradial, or via surgical methods such as a thoracotomy or open-heart surgery.

In some implementations, the device or prosthetic valve 10 may be positioned within a retention area of the delivery system 70 and may be covered with a capsule 75 or may otherwise be retained prior to deployment. In some implementations, the device or prosthetic valve 10 may be self-expanding (e.g., comprise self-expanding portions or features) such that the device expands upon release from the retention area.

In some implementations, the device or prosthetic valve can comprise and/or be formed from a shape memory material. In some implementations, the shape memory material may comprise nitinol or may have other forms in some implementations (such as other forms disclosed herein). In some implementations, the struts of the frames 18, 20, for example, may be made of a shape memory material and may self-expand to the deployed configuration.

In some implementations, the device or prosthetic valve may also take the form of a balloon-expandable prosthetic (e.g., positioned upon an inflatable body or balloon upon entry into the subject’s body, or slid onto an inflatable body or balloon within the subject’s body), or is mechanically expanded, among other forms of deployment.

Referring again to FIG. 6, the delivery system 70 may be advanced to pass into an atrium of a heart and may pass transeptally into another atrium (e.g., from the right atrium to the left atrium) to reach a treatment or implantation site. Such a delivery approach may be utilized for mitral native valve access for example. In some implementations, the delivery system 70 may extend to the right atrium for tricuspid access, or other delivery approaches to other implantation sites may be utilized in some implementations as desired.

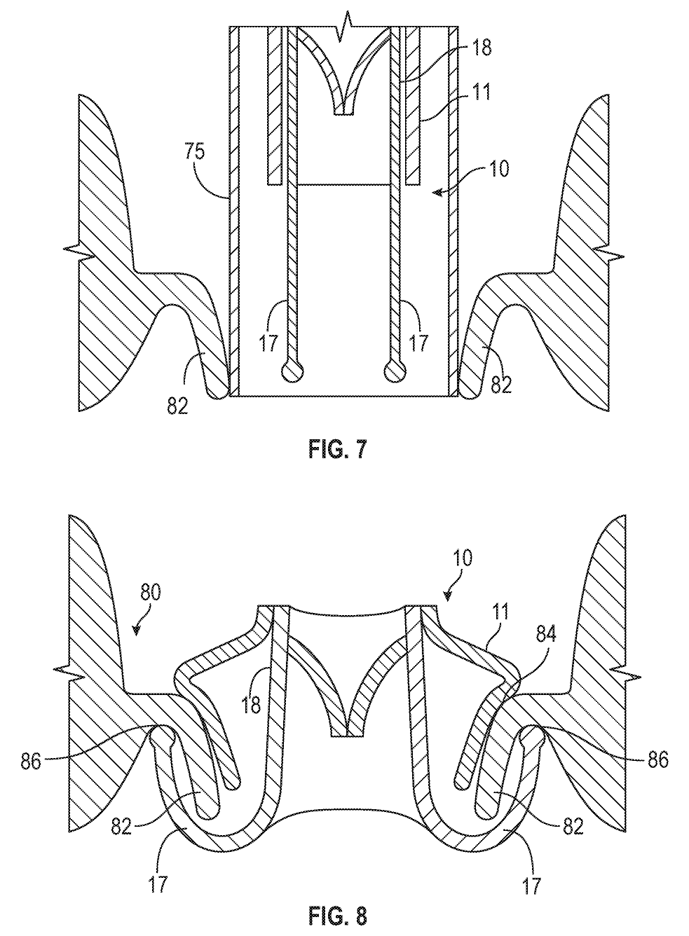

FIG. 7 illustrates the device or prosthetic valve 10 held in a compressed configuration within a capsule 75 of the delivery system 70. The capsule 75 typically has a tubular shape with a hollow interior. When held within the capsule, the prosthetic valve 10 is elongated, with the anchors 17 held in a straightened configuration or elongated configuration. The sealing body 11 (including the outer frame 20) and inner frame 18 each have a reduced diameter and are elongated in FIG. 7. The anchors 17 are advanced and deployed radially outward from the capsule.

FIG. 8 illustrates the device or prosthetic valve 10 deployed to the native valve 80 (e.g., a native mitral valve). The sealing body 11 extends radially outward to contact the inward facing surfaces of the native valve leaflets 82. The anchors 17 hook over native valve leaflets 82 such that the tips of the anchors 17 are positioned radially outward of the native valve leaflets 82.

FIG. 9 illustrates a side view representation of the frame of the device or prosthetic valve 10 upon deployment to a treatment site (e.g., an implantation site, etc.). The native valve leaflets have been excluded from view for clarity.

In some implementations, upon deployment, the shoulder 49 of the outer frame 20 is positioned on a proximal or atrial side of the annulus 84 of the native heart valve.

In some configurations of deployment, the tips of the anchors or tips 23 of the strut arms 21 may be close or may contact the heart tissue 86 on the ventricular side of the heart valve. Such heart tissue 86 is radially outward of the native valve leaflets 82 (as marked in FIG. 8) and forms a ring about the native valve leaflets 82. Such proximity or contact may be undesirable due to impact, pressure, or potential bruising (e.g., pseudo-aneurysm, myocardium puncture, or electrical conduction disturbances) to the heart tissue 86. Further, the anchors are positioned to produce tension in the chords coupled to the leaflets, which may be undesired. The outer frame 20 may seat too high above the annulus.

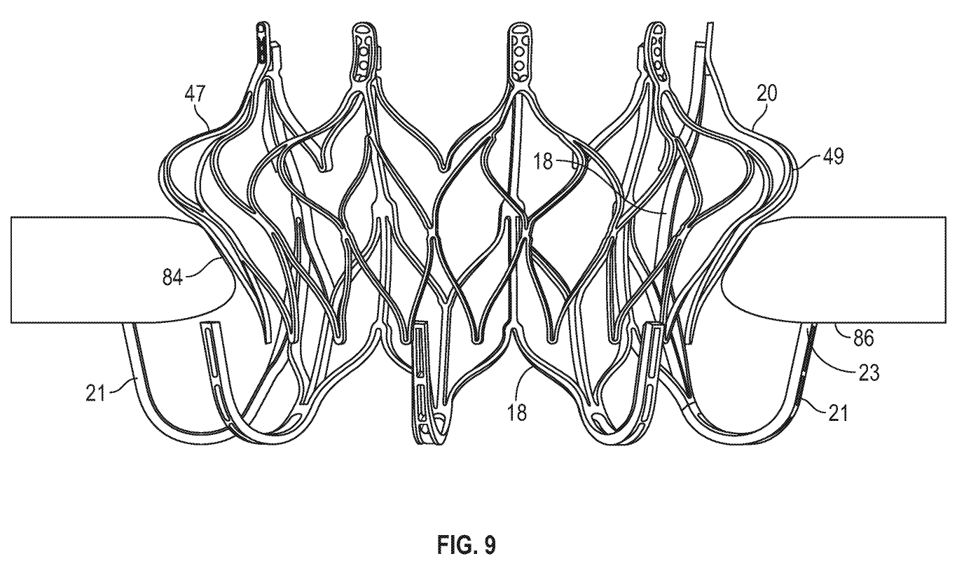

FIG. 10 illustrates an example of an outer frame 90 that may be utilized in some implementations herein.

In some implementations, the device (e.g., the outer frame 90, the inner frame, one or more anchors, etc.) includes a flexibility feature 92.

In some implementations, the flexibility feature allows the outer frame 90 to deflect. In some implementations, the flexibility feature 92 allows the outer frame 90 to deflect to correspondingly allow the inner frame 18 to move axially.

In some implementations, the flexibility feature 92 has a variety of configurations and comprises a portion of the outer frame 90 that is more flexible than an adjacent or other portion of the outer frame 90. In some implementations, the flexibility feature 92, for example, comprises a pattern cut into a strut of the outer frame 90, which comprises an undulating pattern, serpentine pattern, or other form of pattern as desired. In some implementations, the flexibility feature 92 comprises a strain relief feature.

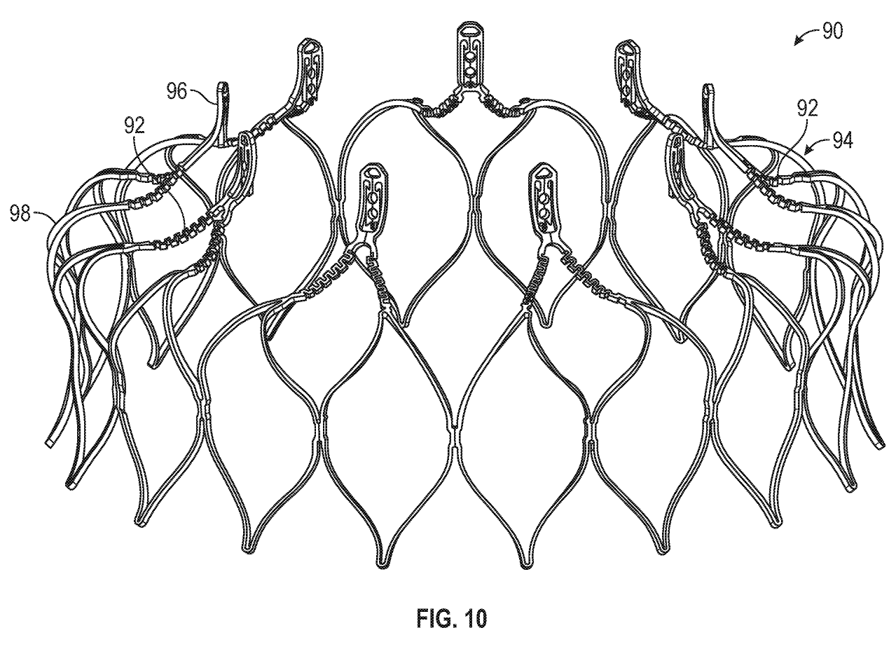

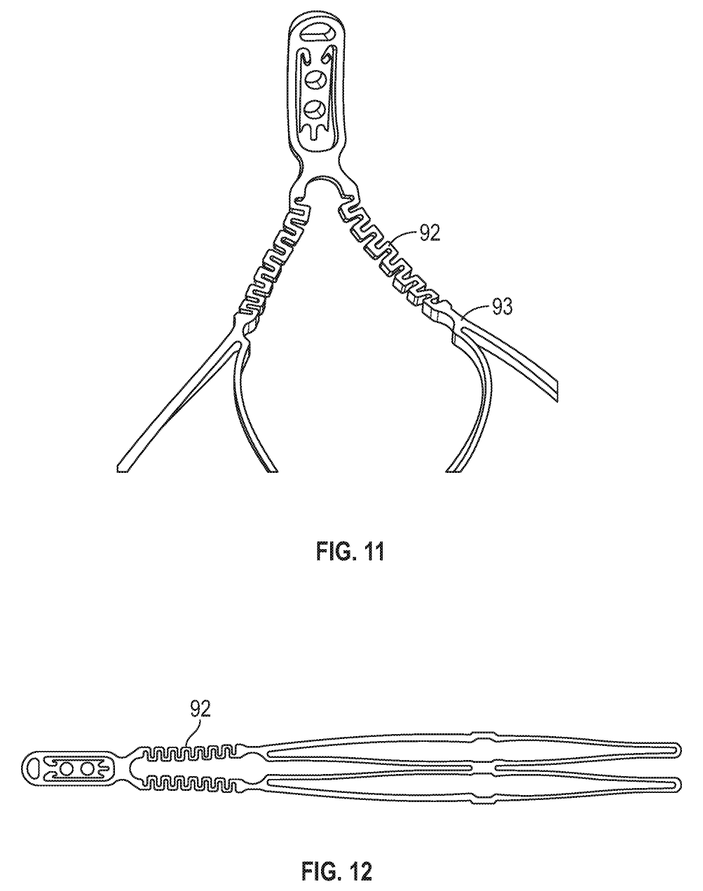

In some implementations, the flexibility feature 92 comprises an undulation in a strut of the outer frame 90. FIG. 11, for example, illustrates a close-up perspective view of the flexibility feature 92 as part of a strut 93 of the outer frame 90. FIG. 12 illustrates a flat pattern of struts of a portion of the outer frame 90 including the flexibility feature 92. In some implementations, the portion of the strut including the flexibility feature 92 is more flexible than an adjacent portion of the strut.

Referring to FIG. 10, the flexibility feature 92 may be positioned at the plateau portion 94 of the outer frame 90, between the inlet end portion 96 and the shoulder 98. The position of the flexibility feature 92 at the plateau portion 94 allows the plateau portion 94 to deflect for axial translation of the inner frame 18 relative to the shoulder 98.

In some implementations, the flexibility feature 92 allows the inlet end portion 96 to move axially relative to the shoulder 98, which correspondingly allows the inner frame 18 to move axially in a distal direction. As such, the axial translation of the inner frame 18 allows the anchors 17 to axially translate relative to the shoulder 98. Although one arrangement is illustrated, it will be understood that other structures are contemplated for allowing an inner frame to move axially within an outer frame for reducing stresses, reducing pressures on surrounding native tissue, and/or for providing a more natural valve motion during beating of the heart.

FIG. 13 illustrates an example flexibility feature 92 during operation according to some implementations. Upon deployment, the flexibility feature 92 allows the outer frame 90 to be more compliant in the axial direction, which allows the inner frame 18 to move axially to a more distal or ventricular position. The outer frame 90 is axially compliant. In some implementations, the corresponding movement of the inner frame 18 allows for a greater clearance 100 or reduced pressure between the tip of the anchor (e.g., the tip 23 of the strut arm 21) and the heart tissue 86. As such, improved clearance and a reduced possibility of adverse pressure or impact upon the heart tissue 86 results (or reduced tension in the chords coupled to the leaflets).

Features disclosed regarding FIG. 10–13 may be utilized alone or in combination with any example disclosed herein.

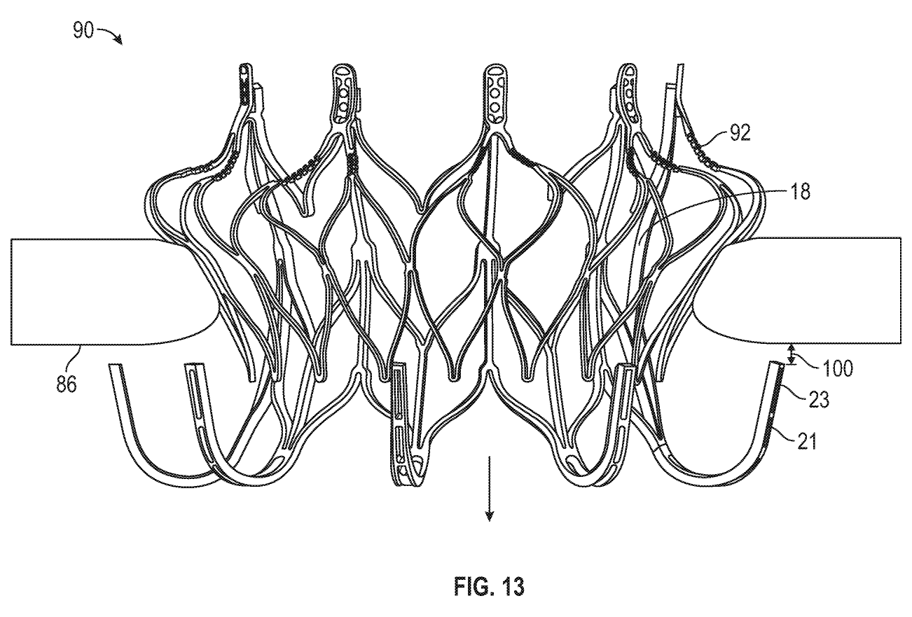

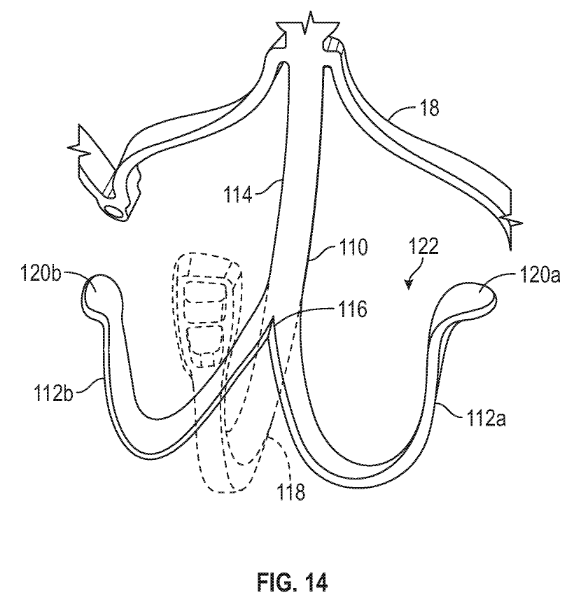

FIG. 14 illustrates another improvement to a prosthetic heart valve wherein an anchor arm 110 is split or forked to form a plurality of prongs 112a, 112b diverging from each other circumferentially.

In some implementations, the anchor arm 110 comprises a strut arm that includes a stem portion 114 that couples directly to the inner frame 18 (e.g., the distal end portion or outflow end portion of the inner frame 18 or support structure 15).

In some implementations, the stem portion 114 extends radially outward to a split portion 116 that forks the anchor arm 110. In some implementations, the split portion 116 divides the plurality of prongs 112a, 112b. In some implementations, the prongs 112a, 112b diverge from each other and from the split portion 116 in circumferentially opposite directions. In some implementations, the stem portion 114 is positioned radially inward of the split portion 116.

In some implementations, the anchor arm includes a curvature that includes a drop loop 118 or lower convex loop. In some implementations, the anchor arm 110 that includes the prongs 112a, 112b follows the hooked contour of an anchor arm as shown in FIG. 13, for example, and similarly includes a drop loop 118. In some implementations, the drop loop 118, however, may be present on the prongs 112a, 112b to follow the hooked contour of the anchor arm shown in FIG. 13.

In some implementations, the split portion 116 accordingly may be positioned inward along the anchor arm 110 relative to the position of the drop loop 118. In some implementations, the split portion 116 may occur at approximately one half a length of the anchor arm 110, yet other positions may be utilized as desired. In some implementations, the split portion may be positioned outward along the anchor arm relative to the drop loop, as shown in FIG. 18, for example.

In some implementations, the prongs 112a, 112b extend from the split portion 116 to respective tips 120a, 120b of the prongs 112a, 112b that are spaced apart and separated from each other. A gap 122 or space extends circumferentially between the tips 120a, 120b.

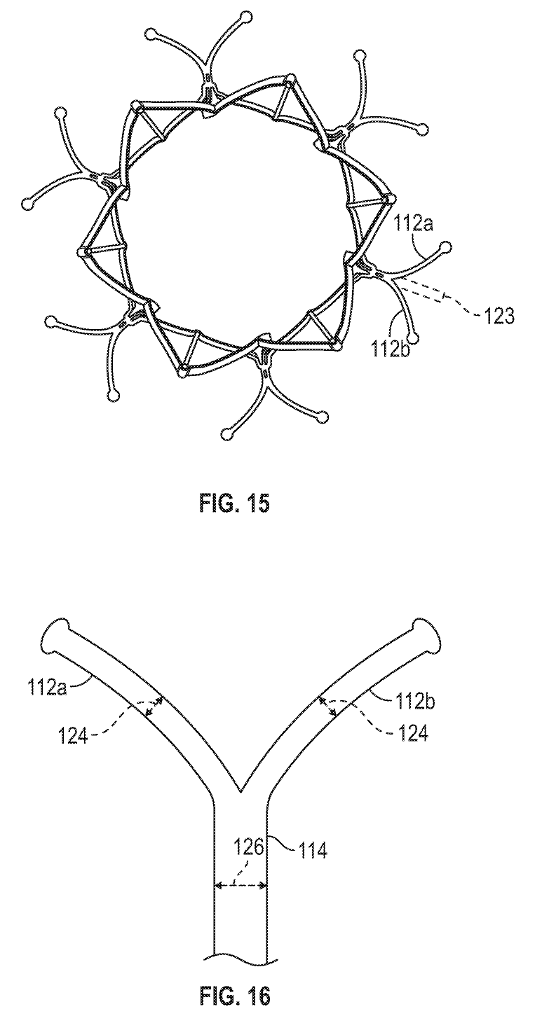

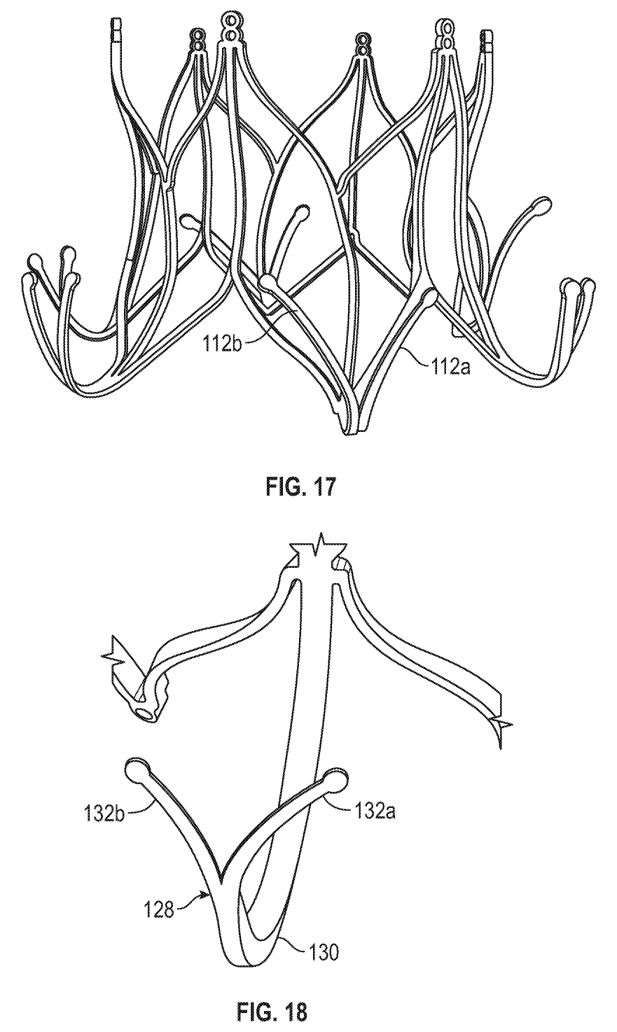

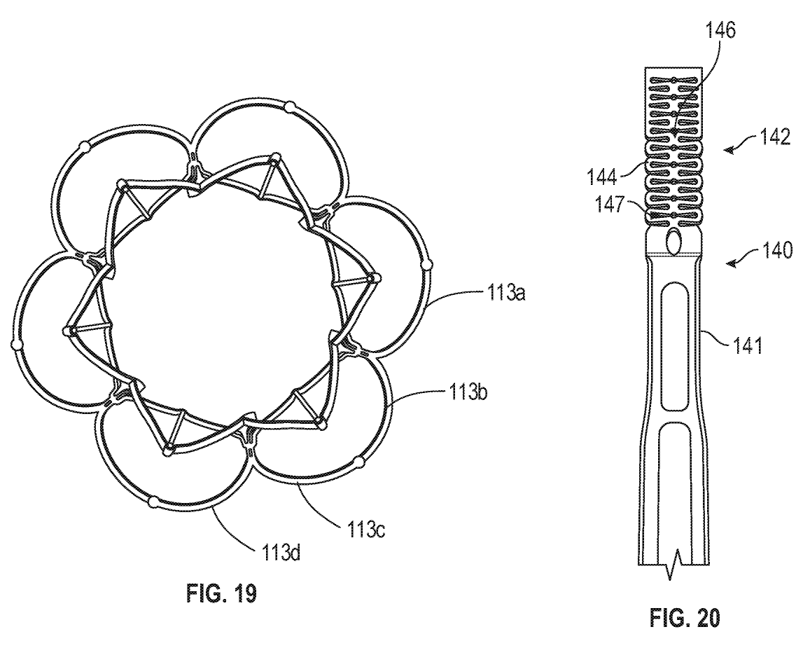

In some implementations, the prongs 112a, 112b diverge circumferentially from each other to cover a larger circumferential area than covered by a single arm configuration as shown in FIG. 3 for example. FIG. 15, for example, illustrates the split of the prongs 112a, 112b covering a larger circumferential area than would be present with a single arm 123 (represented in dashed lines in FIG. 15). In some implementations, the prongs 112a, 112b of the plurality of anchors collectively extend over a circumferential area of at least 80 degrees of the circumference of the prosthetic valve, or at least 90 degrees, or at least 100 degrees, or at least 120 degrees in some implementations. In some implementations, 360-degree coverage is provided as shown in FIG. 19, for example (e.g., with the prong 113b overlapping prong 113c of adjacent anchor, and the prongs 113a, 113d diverging from the respective prongs 113b, 113d).

Referring to FIG. 16, the circumferential width 124 of each of the prongs 112a, 112b is approximately one half of the circumferential width 126 of the stem portion 114. As such, the circumferential width 126 of the stem portion 114 is the same as a combined circumferential width 124 of each of the prongs 112a, 112b. In some implementations, the stem portion 114 has a circumferential width that is greater than a combined circumferential width of the prongs 112a, 112b. Other widths are utilized in some implementations.

FIG. 17 illustrates a side perspective view of an inner frame, with the prongs 112a, 112b diverging from each other circumferentially.

In some implementations, the use of the prongs allows for a lesser number of anchors overall to be utilized, while providing increased circumferential anchoring and stability upon deployment. A reduced risk of missed leaflet capture during deployment results. In some implementations, the prongs 112a, 112b may further be thinner (e.g., a lesser circumferential width) than the stem portion 114, which may increase the flexibility and compliance of each of the prongs 112a, 112b. For example, a reduced possibility of adverse pressure or impact (e.g., pseudo-aneurysm or electrical conduction issues) results. A reduced radial stiffness results, while atrial migration resistance is maintained or enhanced. The prongs 112a, 112b further distribute the anchoring load over a larger surface area than provided with a single anchor arm.

Improved ease in crimping and loading an implant including the prongs results. For example, lower delivery system loading forces results, as opposed to a system in which a greater number of anchor arms is utilized.

FIG. 18 illustrates an implementation in which the split portion 128 is positioned outward of the drop loop 130. A length of the prongs 132a, 132b accordingly is reduced from the length represented in FIG. 14.

In some implementations, the tips of the prongs overlap to close a space between prongs of adjacent anchors. FIG. 19, for example, illustrates the tips of prongs 113b, 113c of adjacent anchors overlapping, which provides 360 degrees of circumferential coverage for the prongs.

In some implementations, the prongs follow the contour of a single anchor arm (e.g., the hooked contour of an anchor arm as shown in FIG. 13) or may have a greater lateral extension as desired.

Features disclosed regarding FIG. 14–19 may be utilized solely or in combination with any other examples disclosed herein.

FIG. 20 illustrates an example in which an anchor arm 140 includes a tip portion 142 with an undulation 144 for increasing a flexibility of the tip portion 142. The undulation 144 comprises a pattern of repeated undulations extending along the tip portion 142 in series.

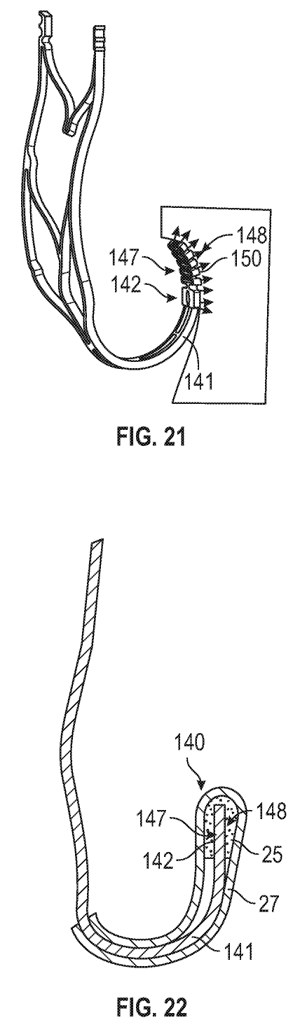

In some implementations, the undulation extends in a circumferential direction or in the circumferential width of the tip portion 142 such that a flat surface 146 remains on the radially inward facing surface 147 of the tip portion 142 and remains on a radially outward facing surface 148 of the tip portion 142 (marked in FIG. 22).

In some implementations, the circumferential width of the tip portion 142 is greater than the radial thickness of the tip portion 142. The tip portion 142 comprises a portion of a strut arm 141 and comprises the end or terminus of the strut arm 141.

In some implementations, the flexible tip portion 142 reduces pressure and impact upon heart tissue if the flexible tip portion 142 contacts the heart tissue. In some implementations, the undulation 144 increases a flexibility of the tip portion 142 in a radially inward direction. The tip portion 142 bends in a radially inward direction. Referring to FIG. 21, for example, the flexible tip portion 142 deflects radially inward to reduce the force upon the heart tissue 150. In some implementations, the flexible tip portion 142 may have a gradual reduction of stiffness towards the terminus of the anchor strut. Uniform flexibility may be provided in some implementations.

Referring to FIG. 22, the strut arm 141 and flexible tip portion 142 are covered with padding or cushioning, such as a pad 25 and sleeve 27 as discussed regarding FIG. 5. In some implementations, the strut arm 141 may be uncovered, with the tip portion 142 directly contacting tissue.

A stress relief anchor tip may result. The tip portion deflects to spread contact force over a larger contact area that is proportional to the anchoring force. Lesser force per unit of contact area results. A greater ability to absorb energy of contact results, and lesser loading forces into a delivery system results.

Features disclosed with respect to FIG. 20–22 may be utilized solely or with any example disclosed herein.

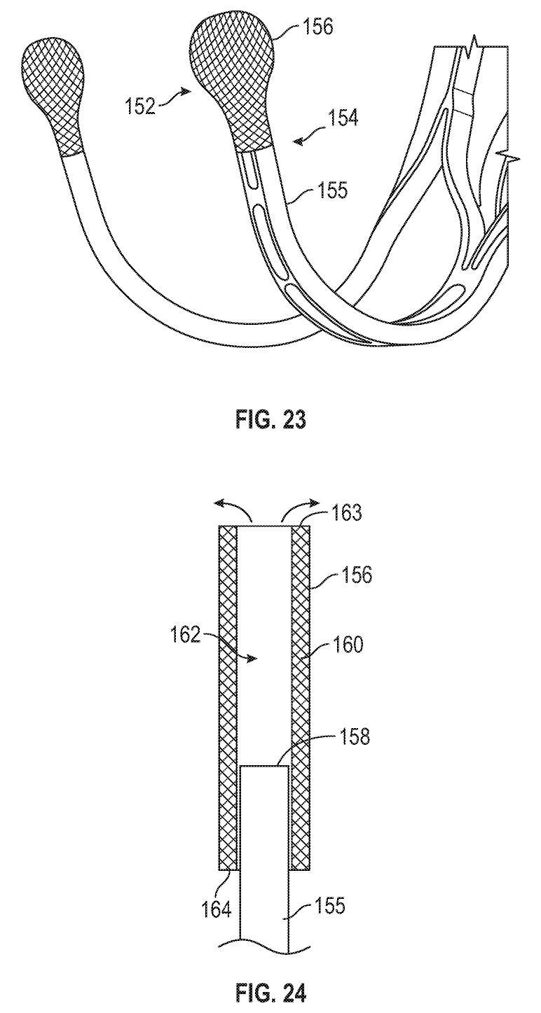

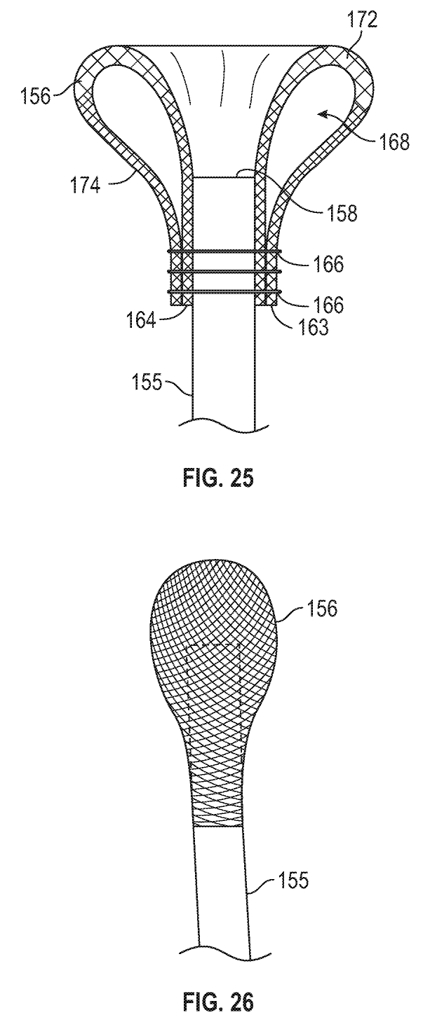

FIG. 23 illustrates an example of a tip portion 152 of an anchor 154 having a compliant braid 156. In some implementations, the compliant braid 156 is positioned at the end surface 158 (marked in FIG. 25) of a strut arm 155 and extends from or protrude from the axial end surface 158. The compliant braid 156 comprises a wire braid or other forms of braid and may be compressible. The wire may comprise a bare wire or may be coated in some implementations.

FIGS. 24 and 25 illustrate an example method or manner of positioning the compliant braid 156 at the axial end surface 158 of the strut arm 155. Referring to FIG. 24, a sleeve 160 of the compliant braid 156 includes an interior lumen 162 that receives the axial end surface 158 of the strut arm 155. The sleeve 160 includes an outer end 163 and an inner end 164, with the inner end 164 surrounding the strut arm 155.

In some implementations, the outer end 163 is inverted and folded outward to be directed back towards the inner end 164 of the sleeve 160. A resulting configuration is shown in FIG. 25. One or more sutures 166 retain the compliant braid 156 in this configuration or the compliant braid 156 may be heat set into a configuration shown in FIG. 25.

In some implementations, the inversion of the outer end 163 forms an annular space 168 that is enclosed by the inversion of the sleeve 160. In some implementations, the annular space 168 is utilized to increase the compliance of the compliant braid 156 by allowing for further movement or flexibility of the compliant braid 156. In some implementations, a cushioning material 170 such as foam (marked in FIG. 28) is positioned within the annular space 168. For example, a ring of the cushioning material 170 is positioned within the annular space 168.

In some implementations, the inversion of the outer end 163 forms a bulb shape for the compliant braid 156. Other shapes are provided as desired. A distal portion 172 of the compliant braid 156 has a braid weave with less density than a proximal portion 174 of the compliant braid 156.

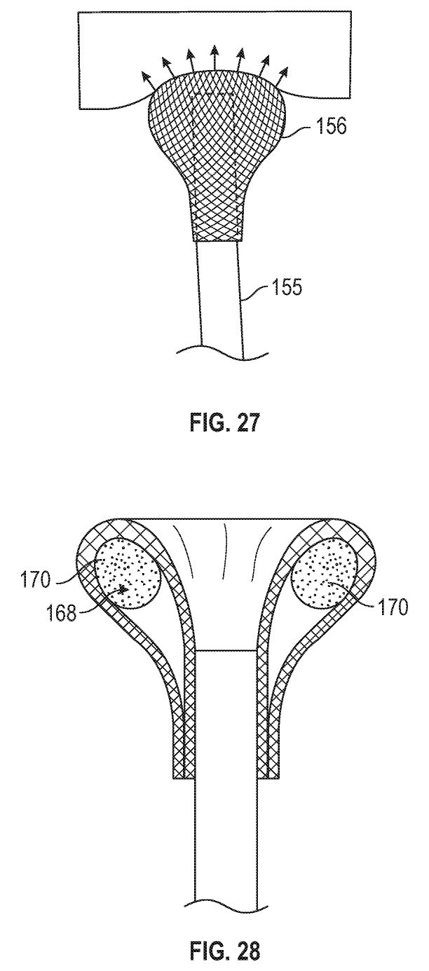

FIG. 26 illustrates a resulting configuration of the compliant braid 156. The compliant braid 156 serves to cushion the strut arm 155 upon a force, as represented in FIG. 27. The compliant braid 156 deforms and spreads upon contact with a portion of a heart, with the spread being proportional to the force. The higher the force, the larger the contact area and thus less pressure per unit of contact area is provided. In some implementations, a compliant braid further conforms to calcification at a treatment or implantation site (e.g., mitral annular calcification (MAC)).

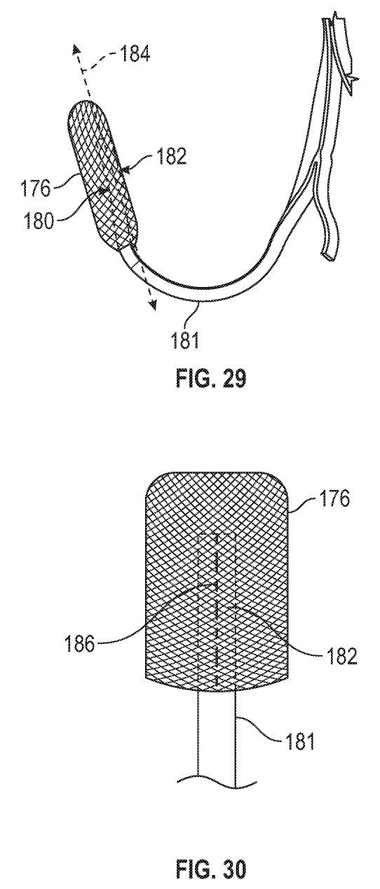

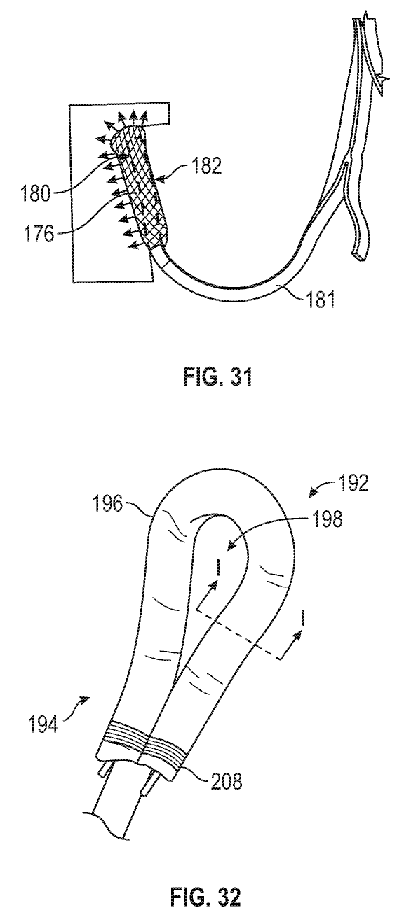

Referring to FIG. 29, in some implementations, a compliant braid 176 is provided that extends along an outward facing surface 180 of a strut arm 181 of an anchor. The compliant braid 176 is offset from the longitudinal axis 184 of the strut arm 181 such that the compliant braid 176 extends further from the radially outward facing surface 180 of the strut arm 181 than from the radially inward facing surface 182 of the strut arm 181. Such a configuration allows the compliant braid 176 to be sutured 186 (as marked in FIG. 30) or otherwise coupled to the strut arm 181 at the inward facing surface 182 of the strut arm 181. The compliant braid 176 cushions the outward facing surface 180 of the strut arm 181 as represented in FIG. 31 for example. The compliant braid 176 further cushions the axial end surface of the strut arm 181 and the inward facing surface 182 of the strut arm 181. The compliant braid 176 covers the radially outward facing surface 180, the radially inward facing surface 182, and the axial end surface of the strut arm.

In some implementations, the compliant braid 176 has a rectangular shape or may have another shape as desired.

The compliant braids disclosed herein can be configured and/or utilized for cushioning against a portion of a heart, to reduce the possibility of puncture, electrical disturbance, or bruising (e.g., pseudo-aneurysm).

Other forms of attachment (e.g., welding) to the strut arm may be utilized in some implementations.

Features disclosed with respect to FIG. 23–31 may be utilized solely or with any example disclosed herein.

FIG. 32 illustrates an example in which a tip portion 192 of the anchor 194 includes a loop 196. The loop 196 comprises a closed loop that bounds an interior space 198 or opening of the loop 196. In some implementations, the loop 196 comprises a compliant loop 196 that increases the flexibility of the anchor at the tip portion 192.

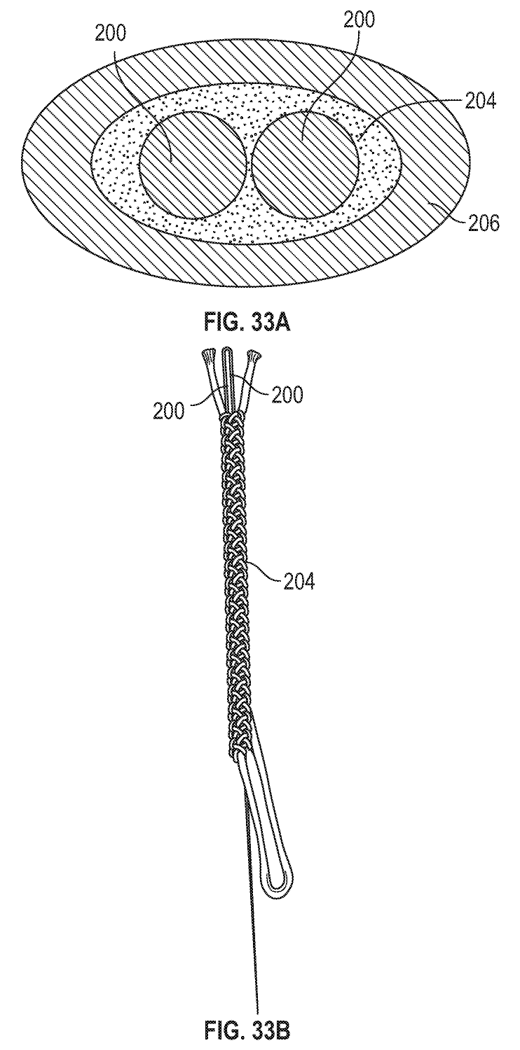

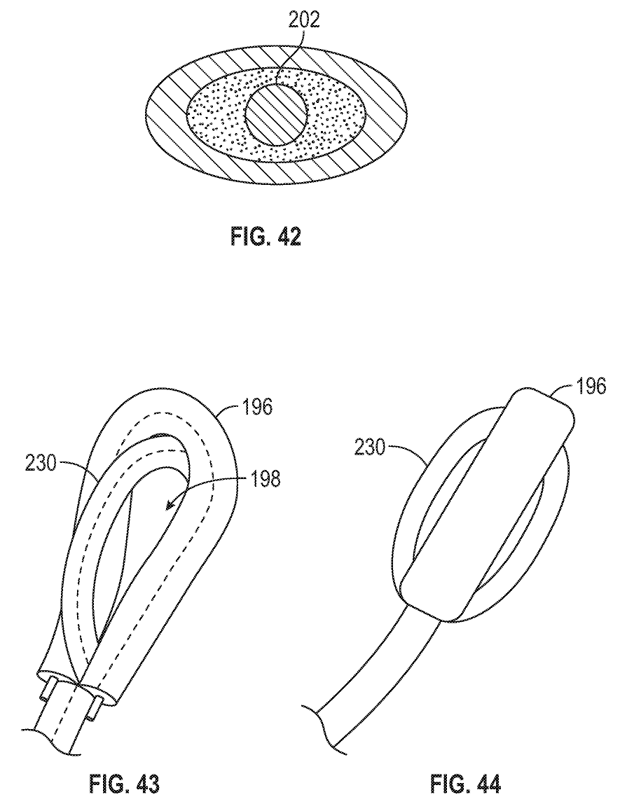

The loop 196 has a variety of compositions as desired. For example, FIG. 33A illustrates a cross-sectional view along line I-I in FIG. 32. The loop 196 includes a core 200 that includes one or more wires extending along a central axis of the loop 196. The core 200 may include multiple wires (e.g., two wires are shown in FIG. 33A). In some implementations, a different number of wires are utilized. FIG. 42, for example, illustrates a single wire or cable utilized as a core 202 for example. The core 200 comprises nitinol wire or wires or another form of material as desired (e.g., shape memory material or another form of material).

In some implementations, a core covering 204 extends over the core 200. The core covering 204 provides a cushion for the core 200. The core covering 204 provides structure to the core 200 and reduces fatigue for the core 200.

In some implementations, the core covering 204 comprises a braid material or other forms of material for providing a cushion upon the core 200. In some implementations, the braid material comprises woven sutures, as represented in FIG. 33B. The core covering 204 comprises a spacer or filler material between the core 200 and the outer cover 206 of the loop 196.

In some implementations, the outer cover 206 extends over the core covering 204 and comprises a sleeve extending over the core covering 204. In some implementations, the outer cover 206 comprises a lubricious coating in some implementations. In some implementations, the outer cover 206, for example, comprises polytetrafluoroethylene (PTFE) or expanded polytetrafluoroethylene (ePTFE) or another form of lubricious coating upon the core covering 204. A lubricious coating aids in reducing abrasion with the native anatomy and allows for deflections to occur. The cross section represented in FIG. 33A comprises continuously extending material that forms a loop, which bounds the interior space 198.

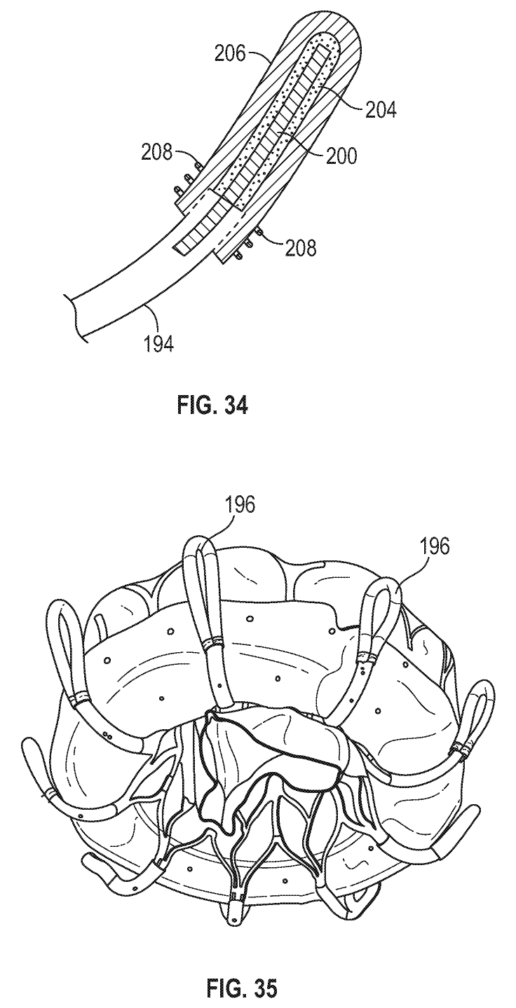

In some implementations, the loop 196 couples to the anchor 194 by the core 200 extending along sides of the anchor 194 and sutures 208 utilized to couple the loop 196 to the anchor 194. FIG. 34, for example, illustrates a side cross-sectional view of an arm of the loop 196.

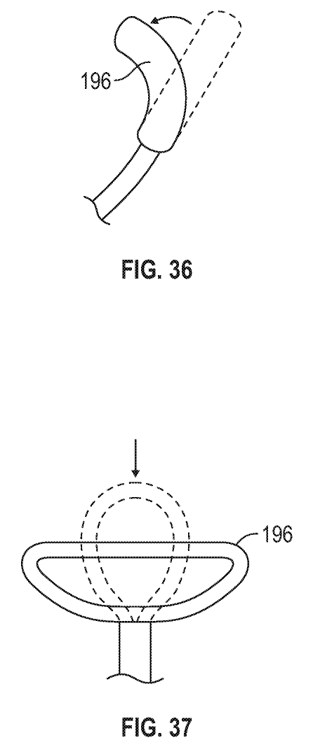

In some implementations, the loops 196 comprise the distal tips of each of the anchors of a prosthetic valve, as represented in FIG. 35, for example. In some implementations, the loops 196 are deflectable in a radially inward direction (as represented in FIG. 36) and may be compressed in a direction along an axis of the respective anchor (as represented in FIG. 37). The loop shape provides resiliency for the anchor tip to return to the loop configuration shown in FIG. 32 for example. In some implementations, the strut arms remain relatively rigid, with the loops being more flexible. As such, the strut arms provide support for the loops, with the loops providing a compliant cushion for use. The size of the loop (as shown in FIG. 37) varies to increase contact surface area and reduce the force per unit of contact area.

Further, the use of compliant loops allows for ventricular remodeling as desired. The construction of the loops further comprises echogenic and/or fluoroscopic indicators, which may indicate whether proper anchoring has occurred (e.g., whether a leaflet has been captured or not).

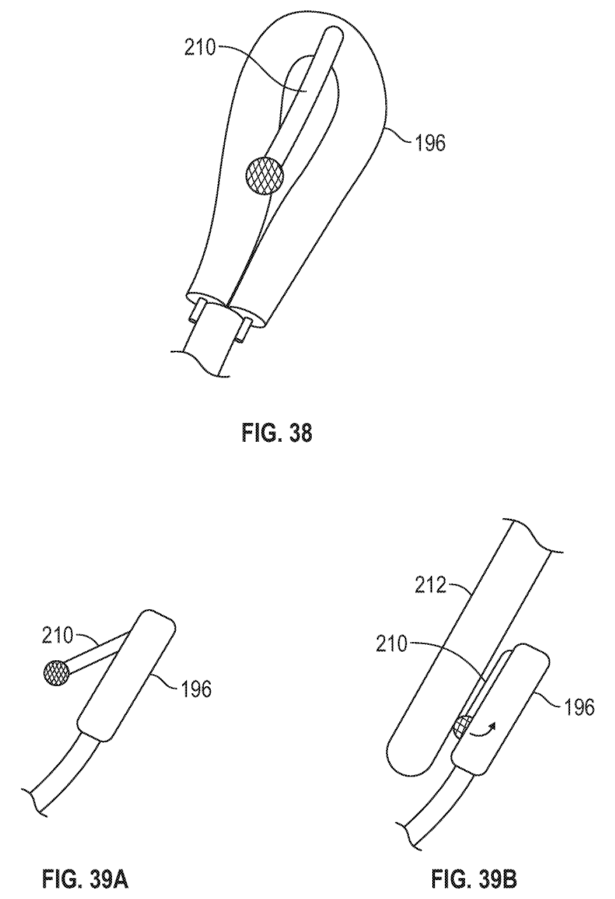

Variations may be used. FIG. 38, for example, illustrates an example in which a capture indicator 210 is positioned at a tip portion of the loop. The capture indicator 210 is in the form of an indicator arm that is coupled to the loop 196.

In some implementations, the indicator 210 protrudes radially inward from the loop 196 (as represented in FIG. 39A). In some implementations, the indicator 210 is pressed radially inward toward the loop 196 by a native valve leaflet 212 to indicate capture of the leaflet 212 by a respective anchor (as represented in FIG. 39B). In some implementations, a lack of movement by the indicator 210 indicates failed capture of the leaflet 212. In some implementations, the indicators comprise echogenic or fluoroscopic indicators, among other forms of indicators.

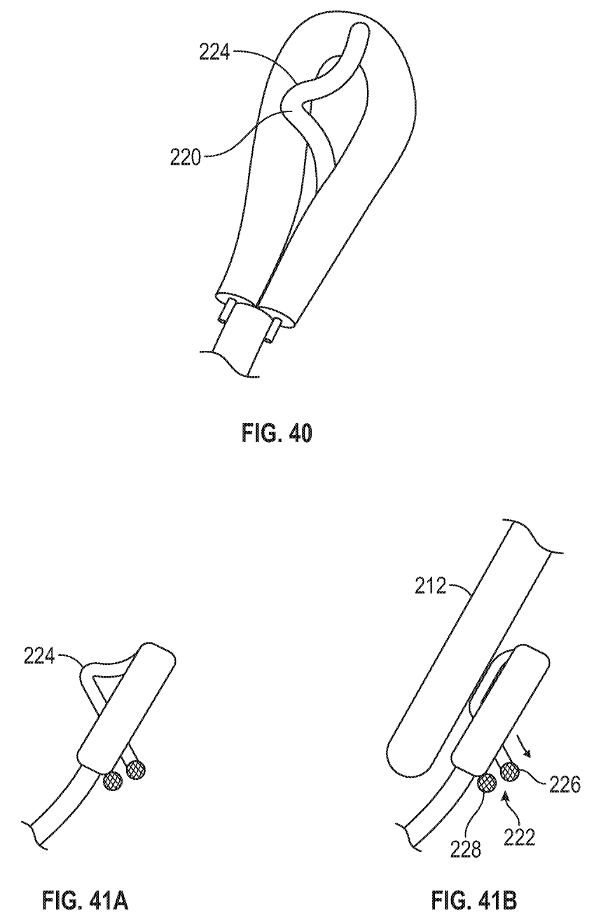

Other forms of indicators may be utilized. FIG. 40 illustrates a configuration of an indicator 220 in which a gap 222 (marked in FIG. 41B) is created upon capture of a leaflet 212 to indicate capture of the leaflet 212. The indicator 220 includes a protruding portion 224 (shown in FIG. 41B) that protrudes radially inward and is pressed by a leaflet 212 (as represented in FIG. 41B) upon capture of the leaflet 212. A gap 222 is formed between a portion 226 of the indicator 220 and a portion 228 of the loop. The portions 226, 228 may comprise respective echogenic or fluoroscopic markers, among other forms of markers.

FIG. 42 illustrates a variation in which a single body core 202 is utilized. The core 202 comprises a single wire or cable as desired. FIG. 43 illustrates a variation in which the loop 196 has another loop 230 pass transverse through the space 198 or opening of the loop 196. The loops 196, 230 form an oblong spheroid configuration as shown in FIGS. 43 and 44.

In some implementations, a filler 240 is provided within the space 198 of the loop 196. The filler 240 comprises a sheet of material that spans the space 198. In some implementations, the filler 240 is provided for tissue ingrowth or has another configuration in some implementations.

FIG. 46 illustrates an example in which a sheet of material 242 is utilized in combination with an indicator 243. The sheet of material 242 extends over the indicator 243 and is utilized for tissue ingrowth or another use as desired. The indicator 243 operates in a similar manner as the indicator 210, as represented in FIGS. 47 and 48.

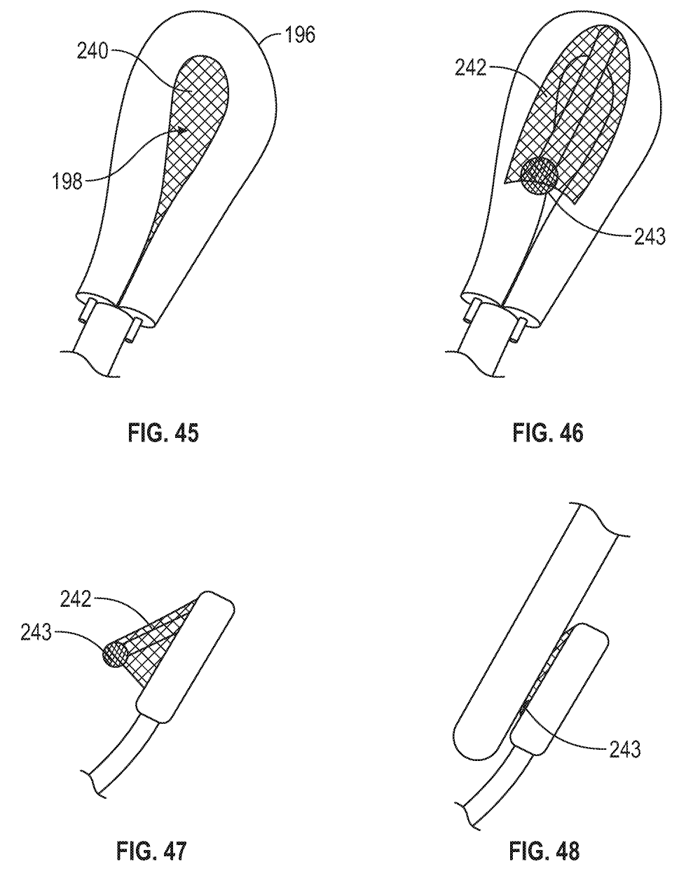

Features disclosed with respect to FIG. 32–48 may be utilized solely or with any example disclosed herein.

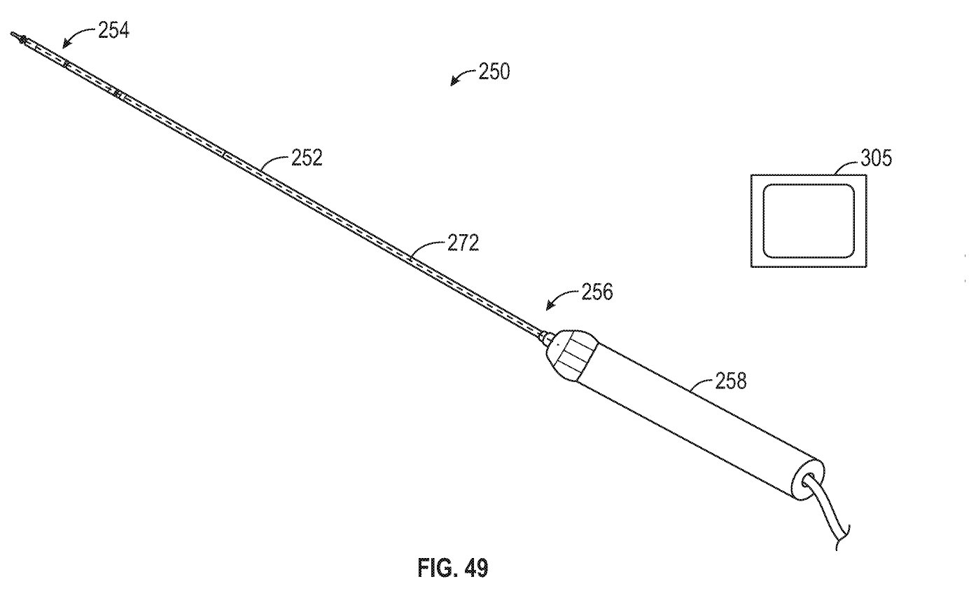

Various delivery system improvements will now be disclosed. In a first example, FIG. 49 illustrates a delivery system 250 having an elongate shaft or catheter shaft 252 having a distal end portion 254 and a proximal end portion 256. The proximal end portion 256 is coupled to a housing in the form of a handle 258 for the catheter shaft 252. The distal end portion 254 includes an implant retention area that retains an implant (such as a prosthetic heart valve) to the catheter shaft 252.

In some implementations, the catheter shaft 252 is flexible for facilitating advancement through the vasculature of a subject, to approach a treatment site or implantation site, in a similar manner as disclosed with respect to FIG. 6 or utilizing a different form of delivery approach (e.g., transapical or directly to the right atrium for tricuspid deployment). The catheter shaft 252 is for advancement of a prosthetic heart valve to a treatment site or implantation site, such as a mitral native valve or a tricuspid native valve, or another native valve (e.g., aortic or pulmonary). The catheter shaft may be steerable to assist with navigation through the vasculature. Steerability may be achieved via incorporation of one or more pullwires that may be actuated via knobs on the handle 258.

In some implementations, the catheter shaft 252 includes a plurality of shafts or sheaths that may overlap each other. In some implementations, the plurality of shafts or sheaths provide a variety of functions, including a capsule sheath that extends over a compressed or crimped implant to retain the implant within the implant retention area, and/or shafts or sheaths that retain the implant in position for deployment to a treatment or implantation site.

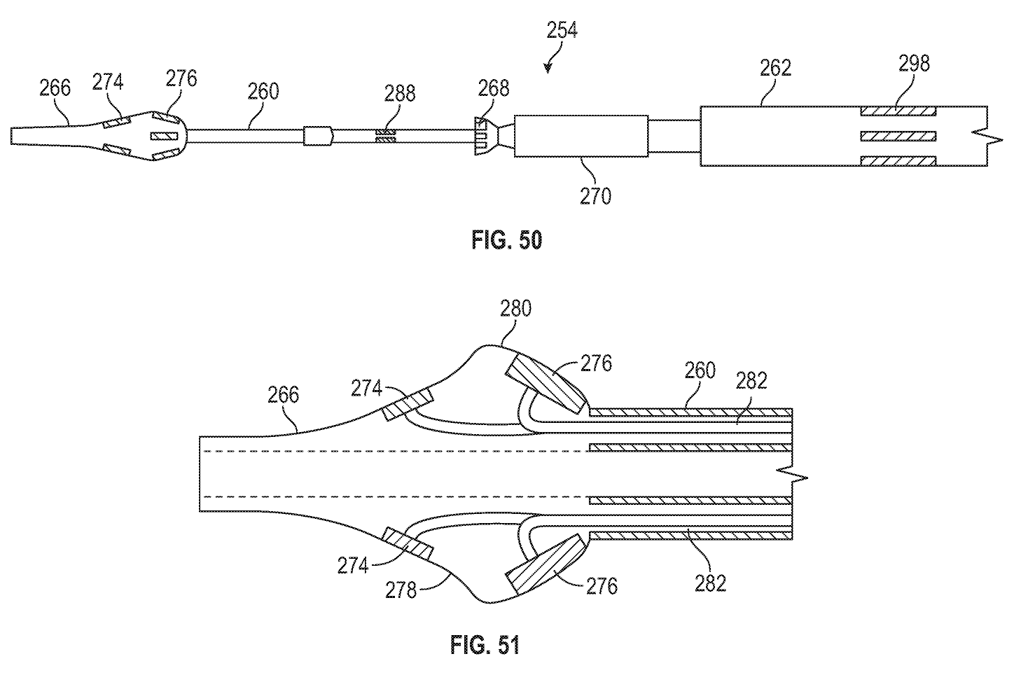

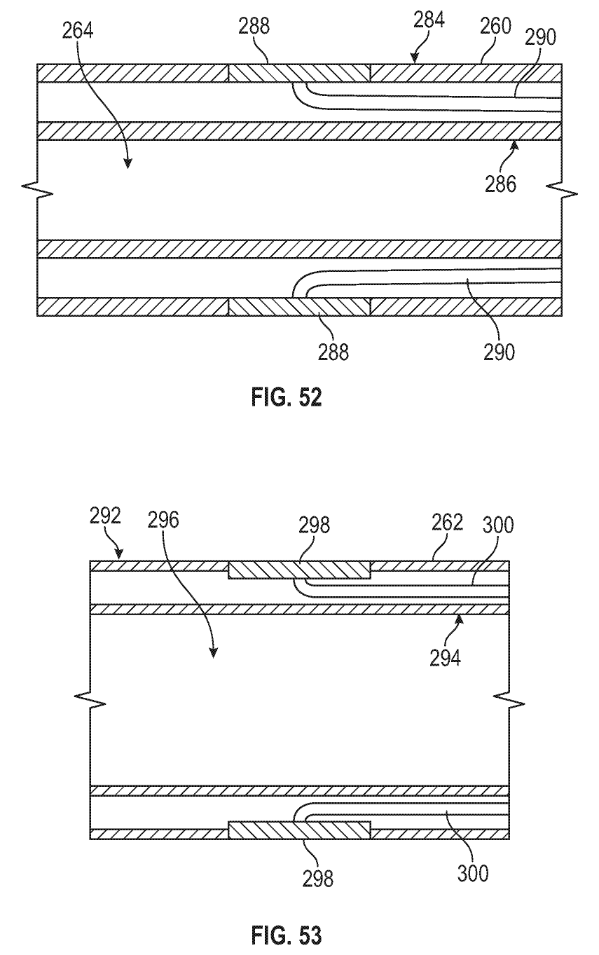

Referring now to FIG. 50, a side view of the distal end portion 254 of the catheter shaft 252 is shown. In some implementations, the shafts or sheaths include a guide wire sheath 260 and a capsule sheath 262. In some implementations, the guide wire sheath 260 includes an interior lumen 264 (marked in FIG. 52) for a guide wire to pass through. In some implementations, a distal end portion of the guide wire sheath 260 is attached to a nose body 266 that forms a tip of the catheter shaft 252.

In some implementations, the capsule sheath 262 comprises a portion of the catheter shaft 252 that covers a retention area for the prosthetic heart valve. In some implementations, the capsule sheath 262 is adapted to advance or retract over the implant (not shown), with retraction of the capsule sheath 262 allowing the implant to expand radially outward and deploy at a treatment or implantation site.

In some implementations, features of the catheter shaft 252 include an implant retainer 268 that is coupled to a shaft or sheath for the catheter shaft 252. The implant retainer 268 retains a portion of the implant. For example, the implant retainer 268 retains proximal end portions of an implant and includes slots to engage with end tab portions of the implant or has another configuration as desired. For example, the implant retainer may include one or more sutures for releasably coupling to an implant. Other configurations of implant retainers may be utilized.

In some implementations, a securement sheath 270 is utilized that extends over a portion of an implant to secure the implant to the catheter shaft 252. The securement sheath 270, for example, extends over a proximal end portion of the implant to retain the implant to the implant retainer 268. Other forms of securement sheaths 270 may be utilized.

The catheter shaft 252 is adapted for extending within the vasculature of a subject. The catheter shaft 252 may passively deflect within the vasculature or a mechanism may be utilized to actively deflect the catheter shaft 252 laterally as desired. Active deflection may allow for controlled navigation within the subject’s vasculature.

In some implementations, one or more components of the delivery system 250 include an imaging system. In some implementations, the imaging system is utilized to image vasculature of the subject’s body from interior of the subject’s vasculature. In some implementations, the imaging may occur upon or during passage of the delivery system 250 through the vasculature of the subject’s body.

In some implementations, the imaging system comprises optical coherence tomography (OCT) or ultrasound imaging (e.g., intravascular ultrasound (IVUS)). Sensors of the imaging system are provided on the delivery system 250 and particularly on the elongate shaft or catheter shaft 252 of the delivery system 250. For example, referring to FIG. 49, signal conduits 272 extend along the catheter shaft 252 for transferring signals from the sensors for receipt and/or processing of the signals.

In some implementations, the sensors of the imaging system are positioned on the nose body 266 in some implementations. For example, referring to FIG. 51, the nose body 266 includes sensors 274, 276 that may be utilized for imaging. In some implementations, the nose body 266 includes a distal surface 278 that is angled to face distally and may include sensors 274. The nose body 266 further includes a proximal surface 280 angled to face proximal and including sensors 276.

In some implementations, the sensors 274 allow for imaging in a distal direction and the sensors 276 allow for imaging in a proximal direction. In some implementations, signal conduits 282 extend from the sensors 276, 274 for transferring the signals from the sensors 276, 274. The signal conduits 282 extend along the guide wire sheath 260 towards the handle of the catheter shaft.

Other positions of sensors may be utilized in some implementations. For example, FIG. 52 illustrates a view of a portion of the guide wire sheath 260 showing the outer surface 284 of the guide wire sheath 260 and the inner surface 286 facing towards the guide wire interior lumen 264. The sensors 288 image radially outward from the outer surface 284 of the guide wire sheath 260. Signal conduits 290 extend from the sensors 288 for transferring the signals from the sensors 288. The signal conduits 290 extend proximally along the guide wire sheath 260 towards the handle of the catheter shaft.

Other positions of sensors may be utilized in some implementations. For example, FIG. 53 illustrates a view of a portion of the capsule sheath 262 showing the outer surface 292 of the capsule sheath 262 and the inner surface 294 facing towards the lumen 296 of the capsule sheath 262. The sensors 298 image radially outward from the outer surface 292 of the capsule sheath 262. Signal conduits 300 extend from the sensors 298 for transferring the signals from the sensors 298. The signal conduits 300 extend proximally along the capsule sheath 262 towards the handle of the catheter shaft.

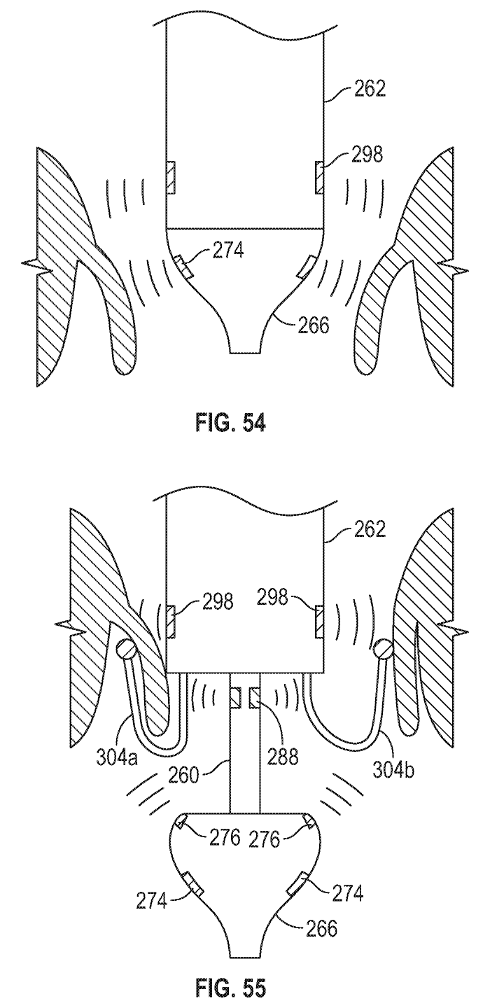

In operation, the imaging system may be utilized to image the implantation site and deployment of the implant to the implantation site. The imaging system may be utilized to image the anchoring, including the hooking of anchors of the implant to the leaflets of the native valve. An example method or procedure is shown in FIGS. 54 and 55.

FIG. 54 illustrates advancement of the distal end portion of the catheter shaft toward the implantation site. One or more of the sensors (e.g., the sensors 274 and/or sensors 298) may be utilized to image the implantation site. Distally facing sensors 274 may be utilized upon approach to the implantation site.

In FIG. 55, the capsule sheath 262 is retracted to allow the anchors 304a, 304b of the implant to expand radially outward. The sensors 276 on the nose body 266 facing proximally are utilized to image the deployment due to the proximal view angle of the sensors 276. The sensors 288 on the guide wire sheath 260 may further be utilized. The implant may be positioned to not impede the view of the capture of the leaflets in such a configuration. In some implementations, a portion of the implant includes openings or apertures to allow for imaging of the capture of the leaflets.

In some implementations, the position of the guide wire sheath 260 is adjusted axially as desired to vary the view provided by the imaging. The guide wire sheath 260 translates axially relative to other portions of the catheter shaft. The variation of the guide wire sheath 260 position varies the position of the nose body 266 and the imaging provided by the nose body 266. As such, the nose body 266 translates axially relative to other portions of the catheter shaft. FIG. 55 illustrates a capture of a leaflet by the anchor 304a, and a failed capture of a leaflet by anchor 304b. The imaging system is utilized to image such capture and failed capture. The imaging plane is set at the plane of capture or missed capture of the leaflets. Other planes (proximal or distal) are set if desired. If desired, the implant may be redeployed as needed for capture of the leaflet by anchor 304b.

In some implementations, the signals from the imaging system are delivered via wired connections to a processor for processing the signals. In some implementations, the signals are processed and displayed on a display 305 as represented in FIG. 49. In some implementations, the ultrasound and/or OCT signals (e.g., optical signals) are processed to provide an image on the display. In some implementations, two-dimensional images are processed and constructed to form a three-dimensional type image on the display 305. Other forms of output may be utilized.

In some implementations, wireless signals from the imaging system are provided. For example, the sensors communicate wirelessly with a processing unit for processing the signals from the sensors.

In a configuration as shown in FIG. 55, the sensors are variably and advantageously moved to vary the view of the deployment procedure. The position of the sensors advantageously allows for improved visualization of capture or missed capture of leaflets, among the position of other features of the deployment procedure.

Various sensor types, locations, and combinations are described herein for purposes of illustration. However, it will be appreciated that the above sensor concepts may be used alone or in combination with other concepts. Various other configurations of imaging systems are contemplated by the scope of this disclosure.

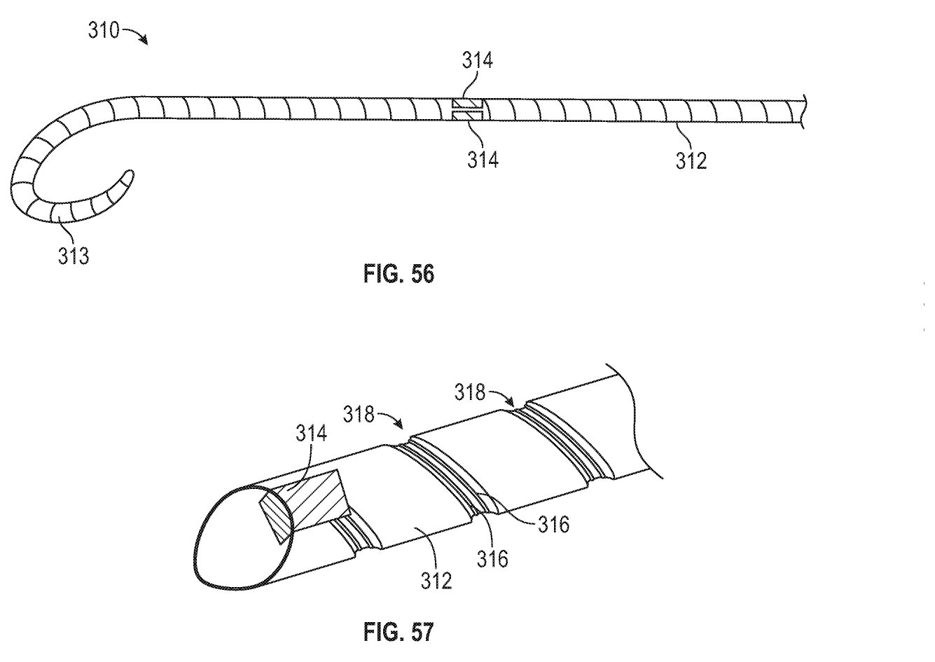

For example, FIG. 56 illustrates an example in which a guide wire 310 includes an imaging system. The guide wire 310 includes a flexible guide wire body 312 with one or more sensors 314 positioned thereon.

In some implementations, the sensors are for optical coherence tomography (OCT) or ultrasound imaging (e.g., intravascular ultrasound (IVUS)), among other forms of imaging.

The guide wire body 312 includes an atraumatic tip 313 at a distal end portion and extends proximally to a proximal end of the guide wire.

FIG. 57 illustrates a close-up perspective view of the guide wire body 312. The sensor conduits 316 extend in a spiral about spiral grooves 318 in the guide wire body 312 among other configurations. The sensor conduits 316 transmit signals from the imaging system to a processor in a similar manner as disclosed regarding other imaging system sensors disclosed herein.

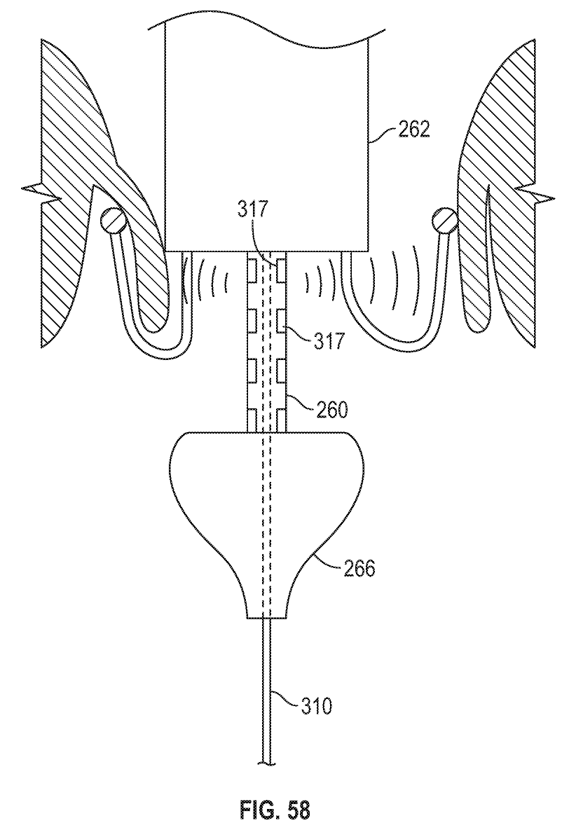

FIG. 58 illustrates an example method or use of the guide wire 310. The guide wire 310 extends within the lumen of the guide wire sheath 260. The imaging system of the guide wire is utilized to image through the guide wire sheath 260 via slots or openings 317 in the guide wire sheath 260. In some implementations, a portion of the guide wire sheath 260 may be essentially transparent to the form of imaging utilized by the imaging sensors.

In an example as shown in FIG. 58, the guide wire 310 is advanced or retracted as desired relative to the guide wire sheath 260 to vary the position of imaging (e.g., the imaging plane) provided by the sensors 314. The position of the imaging system on the guide wire 310 beneficially allows for improved adjustment of the position of the imaging plane. The signals from the imaging system are processed in a similar manner as other imaging systems disclosed herein.

The features of FIGS. 49–58 may be utilized solely or in combination with any other example disclosed herein.

Additional improvements will now be described wherein delivery systems are provided with additional degrees of freedom for facilitating use, operation, and/or implantation of devices (e.g., prosthetic heart valves, treatment devices, etc.) that can be used with any of the various delivery systems described anywhere herein.

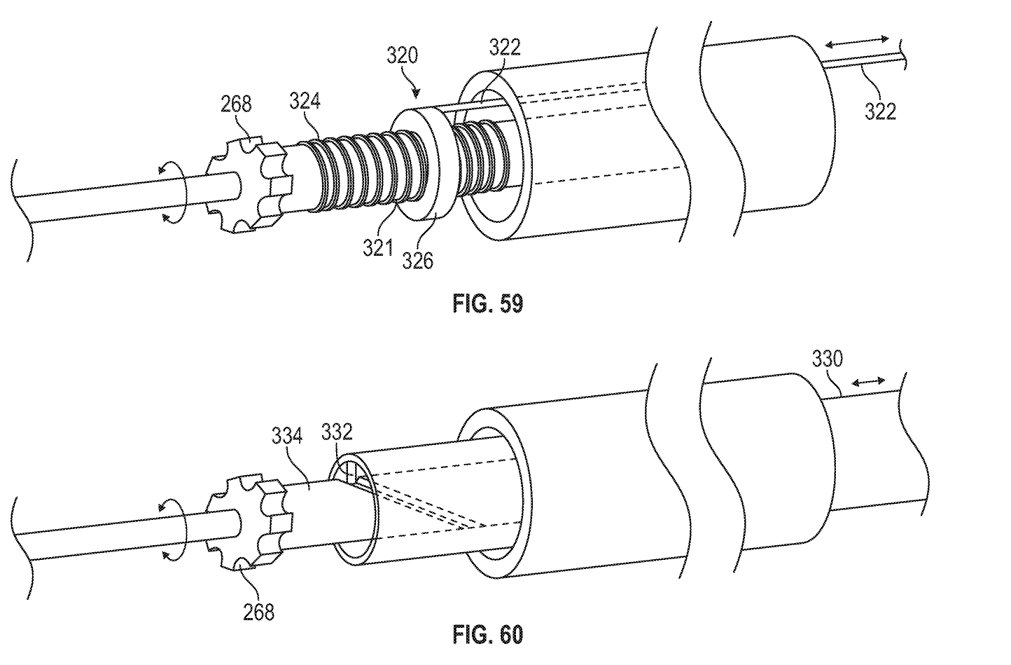

In a first example, FIG. 59 illustrates an actuation mechanism 320 that is advantageously utilized to rotate an implant about an axis of the catheter shaft 252. The actuation mechanism 320 rotates the implant about the axis relative to at least a portion of the catheter shaft 252. The actuation mechanism 320, for example, rotates an implant retainer 268 to produce a corresponding rotation of an implant. The implant retainer 268 is coupled to the implant upon rotation of the implant retainer 268. The implant retainer 268 rotates about the axis of the catheter shaft 252 to correspondingly rotate the implant about the axis.

The actuation mechanism 320 may have a variety of forms. For example, referring to FIG. 59, the actuation mechanism 320 includes a transmission assembly 321. The transmission assembly 321 converts a longitudinal force along a length of the catheter shaft 252 to a rotational force about the axis of the catheter shaft 252. The transmission assembly 321 converts linear or longitudinal movement of an actuator 322 to a rotational movement of the implant retainer 268. The implant retainer 268, for example, is coupled to a gear body 324 or threaded body with an angulation that causes the gear body 324 or threaded body to rotate upon a corresponding gear body 326 or threaded body sliding axially along the gear body 324. As such, the linear movement of the actuator 322 produces a rotation of the implant retainer 268 via the transmission assembly 321.

In some implementations, the actuator 322 comprises a shaft or sheath that extends along the catheter shaft and is operated at a proximal end of the delivery system. In some implementations, the actuator 322 is moved linearly or longitudinally at the proximal end of the delivery system to produce the rotational movement of the implant retainer 268.

FIG. 60 illustrates a configuration in which the actuator 330 comprises a sheath having a pin 332 that engages an angled or cam surface of a body 334. The pin 332 moves linearly or longitudinally to produce a rotation of the body 334. The implant retainer 268 is coupled to the body 334. The linear or longitudinal motion of the actuator 330 accordingly produces the rotation of the implant retainer 268. Other forms of actuators for longitudinal or linear movement are utilized (e.g., pull wires, sutures, shafts, cables, etc.). Other forms of transmissions are utilized as desired for converting longitudinal motion to rotational motion (e.g., gears, cams, screws, etc.). A longitudinal or linear movement of an actuator reduces torque forces that are conveyed along the length of the catheter shaft.

In some implementations, the motion of the actuator occurs due to manual actuation by a user. For example, a user operates an actuator at a proximal end portion of the catheter shaft manually as desired. Forms of manual actuation (e.g., knobs, levers, etc.) are utilized as desired.

In some implementations, a motor is utilized to actuate the actuator. For example, referring to FIG. 61, a motor 340 produces longitudinal movement of the actuator, which produces resulting rotation of the implant retainer 342. The implant retainer 342 is shown to comprise a suture retainer in FIG. 61 (with sutures 343 extending to couple with an implant), although other forms of retainers are utilized in some implementations.

In some implementations, the motor 340 may be operated by a controller 344, which may receive signals from an input device 346. The controller 344 may include a processor and memory therein, to produce instructions to the motor 340 based on the input from the input device 346. In some implementations, direct user control of the motor 340 may be provided.

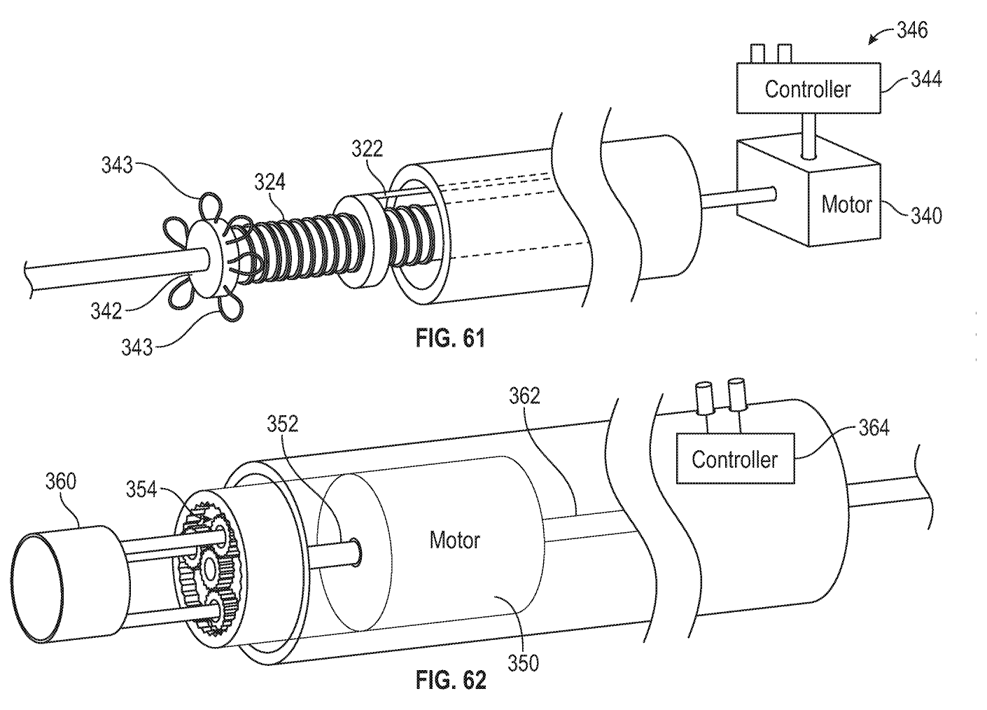

In some implementations, the motor 340 is positioned at the proximal end portion of the catheter shaft (e.g., at or proximate the handle). Referring to FIG. 62, in some implementations, a motor 350 is positioned within the catheter shaft and/or at a distal end portion of the catheter shaft. FIG. 62 illustrates a motor 350 positioned at the distal end portion of the catheter shaft. In some implementations, a motor drive shaft 352 extends to a transmission assembly in the form of one or more gears 354 that are utilized to rotate a retainer sheath 360 for retaining the implant to the implant retainer 268 (not shown) and/or directly rotating an implant retainer 268. In some implementations, the motor 350 operates based on control signals provided by conduits 362 that extend to a controller 364 or otherwise are operated by a user. In some implementations, a power source (e.g., a battery) is positioned with the motor 350 for powering the motor. In some implementations, the power source is positioned at the proximal end portion of the catheter shaft, with power provided via the conduits 362. Various forms of motors (e.g., stepper motors, micromotors, etc.) are utilized in some implementations herein as desired.

The controller, in some implementations, operates based on feedback signals provided from sensors during a deployment procedure. For example, the feedback signals indicate that rotation of the implant is desired, and the controller operates to automatically operate the motor to produce such rotation of the implant.

In some implementations, rotation of other sheaths or shafts of the catheter shaft or other components of the delivery system may be utilized.

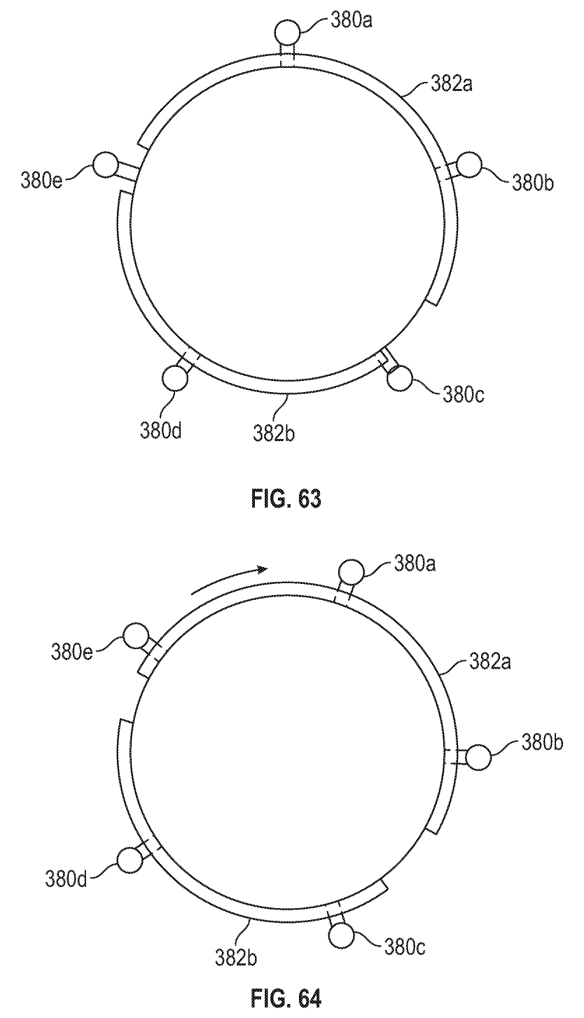

In operation, a rotation of the implant addresses a desired position of the implant. For example, referring to FIG. 63, a top view of anchors 380a through 380e of an implant relative to leaflets 382a, 382b is shown. As illustrated in FIG. 63, the anchors 380e and 380c are not in a desirable position to hook around (i.e., capture) the native leaflets 382a, 382b. More specifically, the anchors 380e and 380c are positioned at gaps or commissures between the native leaflets. Therefore, a slight rotation of the implant may be desired to position the anchors in a more desirable location. As shown in FIG. 64, rotation adjusts the positions of the anchors 380c, 380e relative to the leaflets 382a, 382b. The anchors 380c, 380e shown in FIG. 64 are now positioned adjacent the native leaflets 382a, 382b and are therefore able to anchor more securely thereto.

In some implementations, the rotation of the implant may occur relative to a capsule sheath 262 marked in FIG. 50. In some implementations, the rotation of the implant may occur along with a rotation of the capsule sheath 262.

Other forms of actuation mechanisms may be utilized. For example, an actuation mechanism having an actuator that rotates may be utilized to produce a corresponding rotation of an implant in some implementations.

Other configurations may be utilized in some implementations.

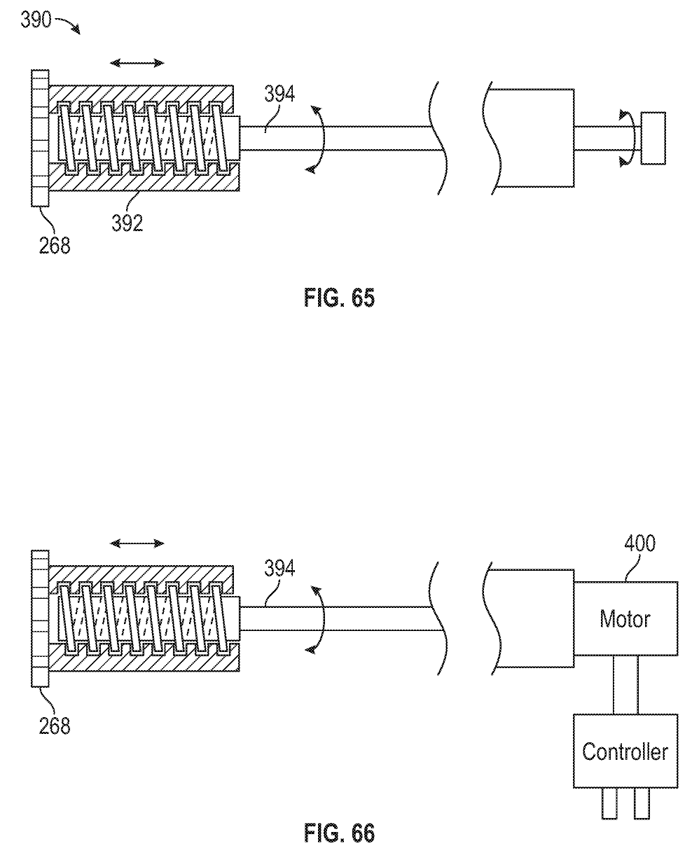

In other examples, actuation mechanisms on delivery systems may be used to vary a depth of the implant, which advantageously provides another degree of freedom during use. For example, referring to FIG. 65, an actuation mechanism 390 includes a transmission assembly 392 for advancing an implant retainer linearly or longitudinally based on a rotation of an actuator 394. The transmission assembly 392 is positioned at a distal end portion of the catheter shaft. The actuator 394 is operable at a proximal end portion of the catheter shaft, for a user to rotate to produce the linear or longitudinal motion of the implant retainer 268. The actuator 394 comprises a sheath or shaft or other device for producing a corresponding rotation at the distal end portion of the catheter shaft.

In some implementations, the transmission assembly 392 comprises a gear system for transmitting a force to vary the depth of the implant. The gear system includes one or more gears (e.g., worm gears) for varying the depth of the implant and producing a corresponding longitudinal or linear movement of the implant retainer 268 as shown in FIG. 65.

Manual rotation of the actuator 394 may be utilized. In some implementations, a motor 400 is utilized to rotate the actuator 394. For example, referring to FIG. 66, a motor 400 is provided at a proximal end portion of the catheter shaft and is adapted to rotate the actuator 394 to produce the linear or longitudinal motion of the implant retainer 268. The motor 400 operates in response to a controller in a similar manner as disclosed herein. The motor causes the variation in the depth of the implant along the axis of the catheter shaft.

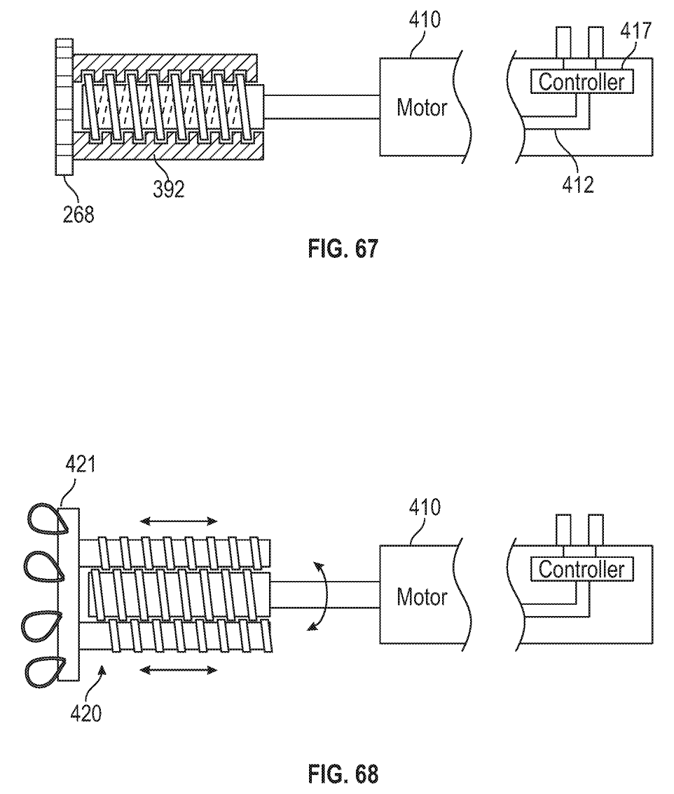

Referring to FIG. 67, in some implementations, a motor 410 is positioned on the catheter shaft and/or at the distal end portion of the catheter shaft. Referring to FIG. 67, the motor 410 couples with control conduits 412, which extend along the catheter shaft to a controller 417. The motor 410 provides a rotational force that produces the linear or longitudinal motion of the implant retainer 268 via the transmission assembly 392. In some implementations, a motor for producing longitudinal movement (e.g., a linear motor) is utilized. Other forms of motors disclosed herein may be utilized.

In some implementations, the form of the implant retainer and the form of the gear system may vary. FIG. 68, for example, illustrate an implant retainer 421 including one or more sutures, and the transmission assembly 420 including a plurality of worm gears.

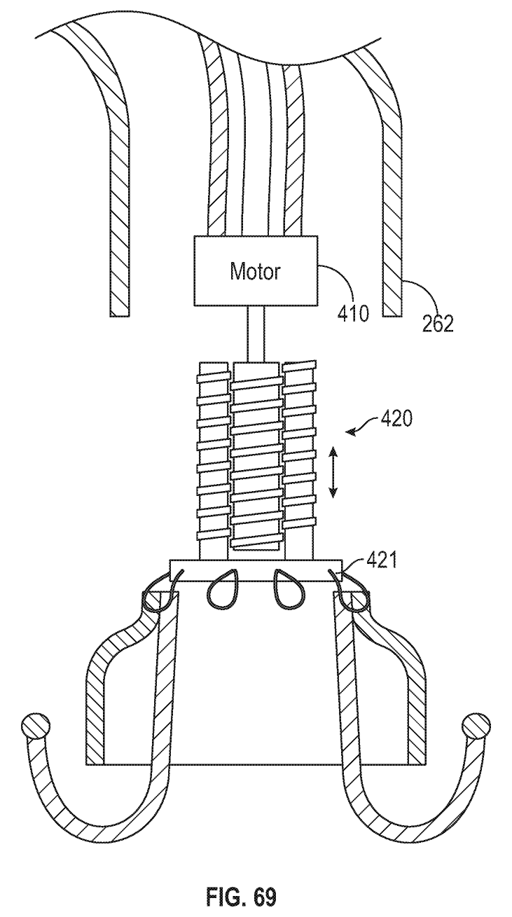

An example use of such a system is represented in FIG. 69. The transmission assembly 420 operates in response to the motor 410 to vary the depth of the implant as desired. In some implementations, the transmission assembly 420 varies the depth of the implant relative to the capsule sheath 262. The depth of the implant is varied to provide the implant at a desired location, which may be the implantation site. For example, the depth of the implant is varied to produce capture of leaflets during a deployment procedure or to otherwise locate the implant at a desired position. Feedback from sensors, utilizing methods as disclosed herein, may be utilized to operate the motor 410 and control the depth as desired.

In some implementations, combinations of mechanisms that vary depth and that rotate an implant may be provided.

The features of FIGS. 59 through 69 may be utilized solely or in combination with any other example disclosed herein.

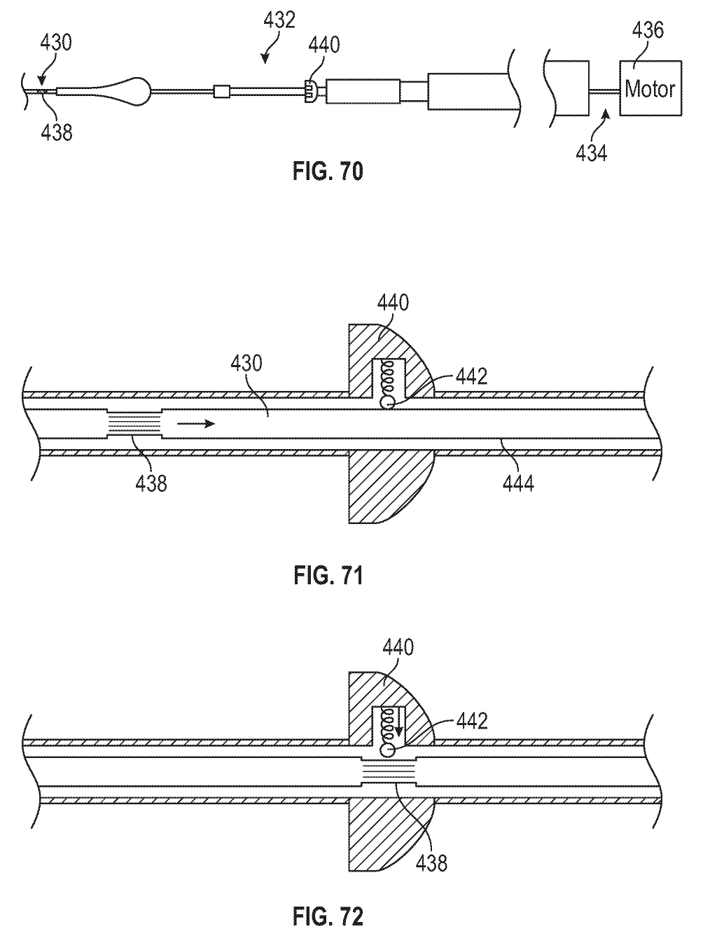

In some implementations, a guide wire is utilized to transmit the actuation of an actuation mechanism. For example, referring to FIG. 70, a guide wire 430 extends along the catheter shaft 432. A proximal end portion 434 of the guide wire 430 is engaged with a motor 436 or may otherwise be manipulated by a user (e.g., manual operation). The guide wire 430 includes a keyed portion or engagement portion (i.e. an engagement feature) 438 for engagement by a component coupled to the implant retainer 440.

In some implementations, the engagement portion 438 is slidable within the catheter shaft longitudinally until engagement by an engagement component 442 coupled to the implant retainer 440. For example, referring to FIG. 71, the engagement component 442 comprises a pin that is biased towards the guide wire 430. In some implementations, the guide wire body 444 comprises a smooth surface or otherwise is not easily engaged by the engagement component 442. At a desired time, the engagement portion 438 is slid to engage the engagement component 442 as shown in FIG. 72 for example.

Referring to FIG. 72, the engagement component 442 engages with the engagement portion 438 and rotation of the guide wire produces corresponding rotation of the implant retainer 440. The guide wire is slid longitudinally to disengage from the engagement component 442 at a desired time.

In some implementations, rotation of the guide wire is produced by a motor 436. In some implementations, manual manipulation of the guide wire may be utilized.

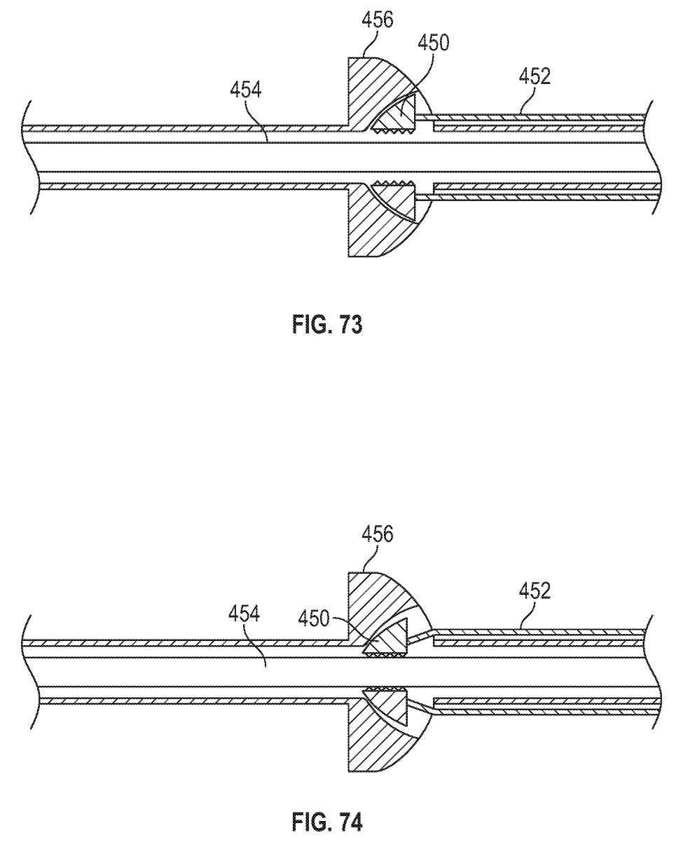

An engagement mechanism is utilized in some implementations. For example, referring to FIG. 73, a grip body 450 is selectively engaged using an actuator 452 that is slid proximally or distally to selectively engage with the guide wire 454. The grip body 450 is pressed forward to cause the grip body 450 to engage an outer surface of the guide wire 454 as shown in FIG. 74. The implant retainer 456 is rotated along with the guide wire 454 as desired. Other forms of engagement may be utilized in some implementations.

The features of FIG. 70–74 may be utilized solely or in combination with any other example disclosed herein.

Variations in the systems and methods disclosed herein are possible and contemplated.

The examples of prosthetic valves may be utilized in a mitral valve as disclosed herein, or may be utilized in other deployment locations such as a native tricuspid valve, or other deployment locations unless stated otherwise. Deployment to aortic or pulmonary valves, or other implantation sites may be utilized.

Features of examples may be utilized solely, or in combination with other features disclosed herein.

Various modifications of the examples disclosed herein may be provided. Features of examples may be modified, substituted, excluded, or combined across examples as desired. Combinations of features across examples may be provided as desired. Combinations of features may be provided across examples with other features of such examples being excluded if desired.

The devices disclosed herein may include treatment devices, repair devices, replacement devices, prosthetic heart valves, and/or other forms of implants, such as stents or filters, or diagnostic devices, among others. The devices may be expandable implants for moving from a compressed or undeployed state to an expanded or deployed state. The devices may be compressible implants adapted to be compressed inward to have a reduced outer profile and to move the implant to the compressed or undeployed state.

Various forms of delivery systems and/or apparatuses may be utilized with the examples disclosed herein. The delivery systems/apparatuses as disclosed herein may be utilized for aortic, mitral, tricuspid, and pulmonary replacement and repair as well. The delivery systems/apparatuses may comprise delivery apparatuses for delivery of other forms of implants, such as stents or filters, or diagnostic devices, among others.

The devices and the systems disclosed herein may be used in transcatheter mitral or tricuspid implantation, as well as aortic valve implantation (TAVI) or replacement of other native heart valves (e.g., pulmonary valves). The delivery apparatuses and the systems disclosed herein may be utilized for transarterial access, including transfemoral access, to a subject’s heart. The delivery apparatuses and systems may be utilized in transcatheter percutaneous procedures, including transarterial procedures, which may be transfemoral. Transapical procedures, among others, may also be utilized. Transjugular procedures may be utilized. Other procedures may be utilized as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein. The examples disclosed herein may comprise systems for implantation within a human body in some implementations.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, along and in various combinations and sub-combinations with one another. The methods, apparatuses, and systems are not limited to any specific aspect or feature or combination thereof, nor do the disclosed examples require that any one or more specific advantages be present or problems be solved. Features, elements, or combinations of one example can be combined into other examples herein.

Example 1: A prosthetic heart valve for deployment to a native heart valve, the prosthetic heart valve comprising: a support structure having an inlet end portion and an outlet end portion and a passageway, the support structure including: an inner frame having an inlet end portion and an outlet end portion, and/or an outer frame including an inlet end portion that is coupled to the inlet end portion of the inner frame, the outer frame including a plateau portion that extends radially outward from the inlet end portion of the outer frame to a shoulder of the outer frame, and/or the outer frame including an axially extending portion that extends axially from the shoulder to an outlet end portion of the outer frame, the shoulder angling the plateau portion relative to the axially extending portion, and/or the plateau portion including a flexibility feature that allows the plateau portion to deflect for axial translation of the inner frame relative to the shoulder; and/or a valve portion positioned within the passageway of the support structure, wherein the valve portion comprises a plurality of prosthetic valve leaflets, wherein the valve portion permits flow of blood through the passageway in one direction for replacing the function of the native heart valve.

Example 2: The prosthetic heart valve of any example herein, in particular Example 1, wherein the flexibility feature comprises a portion of a strut of the outer frame that is more flexible than an adjacent portion of the strut.

Example 3: The prosthetic heart valve of any example herein, in particular Example 1 or Example 2, wherein the flexibility feature comprises an undulation of a strut of the outer frame.

Example 4: The prosthetic heart valve of any example herein, in particular Examples 1–3, further comprising one or more anchors coupled to the outlet end portion of the inner frame, the one or more anchors each having a hook shape.

Example 5: The prosthetic heart valve of any example herein, in particular Example 4, wherein the flexibility feature allows the plateau portion to deflect for axial translation of the one or more anchors relative to the shoulder.

Example 6: The prosthetic heart valve of any example herein, in particular Example 5, wherein each of the one or more anchors has a tip, and/or the axial translation of the one or more anchors relative to the shoulder moves the tip of the respective anchor relative to the shoulder.

Example 7: The prosthetic heart valve of any example herein, in particular Examples 1–6, wherein the flexibility feature allows the plateau portion to deflect for axial translation of the inlet end portion of the inner frame relative to the shoulder.

Example 8: The prosthetic heart valve of any example herein, in particular Examples 1–7, wherein the outer frame is self-expanding.

Example 9: The prosthetic heart valve of any example herein, in particular Examples 1–8, further comprising a sealing skirt coupled to the outer frame.

Example 10: The prosthetic heart valve of any example herein, in particular Examples 1–9, wherein the prosthetic heart valve is a replacement mitral heart valve or a replacement tricuspid heart valve.

Example 11: A method comprising: deploying a prosthetic heart valve to a native heart valve, the prosthetic heart valve including: a support structure having an inlet end portion and an outlet end portion and a passageway, the support structure including: an inner frame having an inlet end portion and an outlet end portion, and/or an outer frame including an inlet end portion that is coupled to the inlet end portion of the inner frame, the outer frame including a plateau portion that extends radially outward from the inlet end portion of the outer frame to a shoulder of the outer frame, and/or the outer frame including an axially extending portion that extends axially from the shoulder to an outlet end portion of the outer frame, the shoulder angling the plateau portion relative to the axially extending portion, and/or the plateau portion including a flexibility feature that allows the plateau portion to deflect for axial translation of the inner frame relative to the shoulder, and/or a valve portion positioned within the passageway of the support structure, wherein the valve portion comprises a plurality of prosthetic valve leaflets, wherein the valve portion permits flow of blood through the passageway in one direction for replacing the function of the native heart valve.

Example 12: The method of any example herein, in particular Example 11, wherein the flexibility feature comprises a portion of a strut of the outer frame that is more flexible than an adjacent portion of the strut.

Example 13: The method of any example herein, in particular Example 11 or Example 12, wherein the flexibility feature comprises an undulation of a strut of the outer frame.

Example 14: The method of any example herein, in particular Examples 11–13, wherein one or more anchors are coupled to the outlet end portion of the inner frame, the one or more anchors each having a hook shape.

Example 15: The method of any example herein, in particular Example 14, wherein the flexibility feature allows the plateau portion to deflect for axial translation of the one or more anchors relative to the shoulder.

Example 16: The method of any example herein, in particular Example 15, wherein each of the one or more anchors has a tip, and/or the axial translation of the one or more anchors relative to the shoulder moves the tip of the respective anchor relative to the shoulder.

Example 17: The method of any example herein, in particular Examples 11–16, wherein the flexibility feature allows the plateau portion to deflect for axial translation of the inlet end portion of the inner frame relative to the shoulder.

Example 18: The method of any example herein, in particular Examples 11–17, wherein the outer frame is self-expanding.

Example 19: The method of any example herein, in particular Examples 11–18, wherein a sealing skirt is coupled to the outer frame.

Example 20: The method of any example herein, in particular Examples 11–19, wherein the native heart valve is a mitral heart valve or a tricuspid heart valve.

Example 21: A prosthetic heart valve for deployment to a native heart valve, the prosthetic heart valve comprising: a support structure having an inlet end portion and an outlet end portion and a passageway; a valve portion positioned within the passageway of the support structure, wherein the valve portion comprises a plurality of prosthetic valve leaflets, wherein the valve portion permits flow of blood through the passageway in one direction for replacing the function of the native heart valve; and/or one or more anchors extending radially outward from the support structure, the one or more anchors each having a hook shape and/or including an anchor arm that is forked to form a plurality of prongs diverging from each other circumferentially.

Example 22: The prosthetic heart valve of any example herein, in particular Example 21, wherein the anchor arm includes a split portion that forks the anchor arm, and/or a stem portion positioned radially inward of the split portion, the stem portion being coupled to the outlet end portion of the support structure.

Example 23: The prosthetic heart valve of any example herein, in particular Example 22, wherein the stem portion has a circumferential width that is the same as or greater than a combined circumferential width of each of the plurality of prongs.

Example 24: The prosthetic heart valve of any example herein, in particular Example 22 or Example 23, wherein the one or more anchors include a plurality of the anchors, and/or the plurality of prongs of the plurality of the anchors extend over at least about 120 degrees of a circumference of the prosthetic heart valve.

Example 25: The prosthetic heart valve of any example herein, in particular Examples 21–24, wherein the one or more anchors include a plurality of the anchors, wherein one of the plurality of prongs overlaps one of the plurality of prongs of an adjacent one of the plurality of anchors.

Example 26: The prosthetic heart valve of any example herein, in particular Examples 21–25, wherein the anchor arm includes a drop loop and/or a split portion that forks the anchor arm, the split portion being positioned outward of the drop loop.

Example 27: The prosthetic heart valve of any example herein, in particular Examples 21–26, wherein the support structure includes an inner frame having an inlet end portion and/or an outlet end portion, and/or an outer frame including an inlet end portion that is coupled to the inlet end portion of the inner frame.

Example 28: The prosthetic heart valve of any example herein, in particular Example 27, wherein the one or more anchors are coupled to the outlet end portion of the inner frame.

Example 29: The prosthetic heart valve of any example herein, in particular Example 27 or Example 28, wherein the outer frame is self-expanding.

Example 30: The prosthetic heart valve of any example herein, in particular Examples 21–29, wherein the prosthetic heart valve is a replacement mitral heart valve or a replacement tricuspid heart valve.

Example 31: A method comprising: deploying a prosthetic heart valve to a native heart valve, the prosthetic heart valve including: a support structure having an inlet end portion and an outlet end portion and a passageway, a valve portion positioned within the passageway of the support structure, wherein the valve portion comprises a plurality of prosthetic valve leaflets, wherein the valve portion permits flow of blood through the passageway in one direction for replacing the function of the native heart valve, and/or one or more anchors extending radially outward from the support structure, the one or more anchors each having a hook shape and/or including an anchor arm that is forked to form a plurality of prongs diverging from each other circumferentially.

Example 32: The method of any example herein, in particular Example 31, wherein the anchor arm includes a split portion that forks the anchor arm, and/or a stem portion positioned radially inward of the split portion, the stem portion being coupled to the outlet end portion of the support structure.

Example 33: The method of any example herein, in particular Example 32, wherein the stem portion has a circumferential width that is the same as or greater than a combined circumferential width of each of the plurality of prongs.

Example 34: The method of any example herein, in particular Example 32 or Example 33, wherein the one or more anchors include a plurality of the anchors, and/or the plurality of prongs of the plurality of the anchors extend over at least about 120 degrees of a circumference of the prosthetic heart valve.

Example 35: The method of any example herein, in particular Examples 31–34, wherein the one or more anchors include a plurality of the anchors, wherein one of the plurality of prongs overlaps one of the plurality of prongs of an adjacent one of the plurality of anchors.

Example 36: The method of any example herein, in particular Examples 31–35, wherein the anchor arm includes a drop loop and/or a split portion that forks the anchor arm, the split portion being positioned outward of the drop loop.

Example 37: The method of any example herein, in particular Examples 31–36, wherein the support structure includes an inner frame having an inlet end portion and/or an outlet end portion, and/or an outer frame including an inlet end portion that is coupled to the inlet end portion of the inner frame.

Example 38: The method of any example herein, in particular Example 37, wherein the one or more anchors are coupled to the outlet end portion of the inner frame.

Example 39: The method of any example herein, in particular Example 37 or Example 38, wherein the outer frame is self-expanding.

Example 40: The method of any example herein, in particular Examples 31–39, wherein the native heart valve is a mitral heart valve or a tricuspid heart valve.

Example 41: A prosthetic heart valve for deployment to a native heart valve, the prosthetic heart valve comprising: a support structure having an inlet end portion and an outlet end portion and a passageway; a valve portion positioned within the passageway of the support structure, wherein the valve portion comprises a plurality of prosthetic valve leaflets, wherein the valve portion permits flow of blood through the passageway in one direction for replacing the function of the native heart valve; and/or one or more anchors extending radially outward from the support structure, the one or more anchors each having a hook shape and/or including a tip portion with an undulation for increasing a flexibility of the tip portion.

Example 42: The prosthetic heart valve of any example herein, in particular Example 41, wherein each of the anchors includes a strut arm, the strut arm having the tip portion, the tip portion of the strut arm having a circumferential width that is greater than a radial thickness of the tip portion of the strut arm.

Example 43: The prosthetic heart valve of any example herein, in particular Example 41 or Example 42, wherein the undulation extends in a circumferential direction on the tip portion.

Example 44: The prosthetic heart valve of any example herein, in particular Examples 41–43, wherein the undulation increases a flexibility of the tip portion in a radially inward direction.

Example 45: The prosthetic heart valve of any example herein, in particular Examples 41–44, wherein the undulation allows the tip portion to bend in a radially inward direction.

Example 46: The prosthetic heart valve of any example herein, in particular Examples 41–45, wherein the tip portion has a gradual reduction of stiffness towards a terminus of the respective anchor.

Example 47: The prosthetic heart valve of any example herein, in particular Examples 41–46, wherein the support structure includes an inner frame having an inlet end portion and an outlet end portion, and/or an outer frame including an inlet end portion that is coupled to the inlet end portion of the inner frame.

Example 48: The prosthetic heart valve of any example herein, in particular Example 47, wherein the one or more anchors are coupled to the outlet end portion of the inner frame.

Example 49: The prosthetic heart valve of any example herein, in particular Example 47 or Example 48, wherein the outer frame is self-expanding.

Example 50: The prosthetic heart valve of any example herein, in particular Examples 41–49, wherein the prosthetic heart valve is a replacement mitral heart valve or a replacement tricuspid heart valve.

Example 51: A method comprising: deploying a prosthetic heart valve to a native heart valve, the prosthetic heart valve including: a support structure having an inlet end portion and an outlet end portion and a passageway, a valve portion positioned within the passageway of the support structure, wherein the valve portion comprises a plurality of prosthetic valve leaflets, wherein the valve portion permits flow of blood through the passageway in one direction for replacing the function of the native heart valve, and/or one or more anchors extending radially outward from the support structure, the one or more anchors each having a hook shape and/or including a tip portion with an undulation for increasing a flexibility of the tip portion.

Example 52: The method of any example herein, in particular Example 51, wherein each of the anchors includes a strut arm, the strut arm having the tip portion, the tip portion of the strut arm having a circumferential width that is greater than a radial thickness of the tip portion of the strut arm.

Example 53: The method of any example herein, in particular Example 51 or Example 52, wherein the undulation extends in a circumferential direction on the tip portion.

Example 54: The method of any example herein, in particular Examples 51–53, wherein the undulation increases a flexibility of the tip portion in a radially inward direction.

Example 55: The method of any example herein, in particular Examples 51–54, wherein the undulation allows the tip portion to bend in a radially inward direction.

Example 56: The method of any example herein, in particular Examples 51–55, wherein the tip portion has a gradual reduction of stiffness towards a terminus of the respective anchor.

Example 57: The method of any example herein, in particular Examples 51–56, wherein the support structure includes an inner frame having an inlet end portion and an outlet end portion, and/or an outer frame including an inlet end portion that is coupled to the inlet end portion of the inner frame.

Example 58: The method of any example herein, in particular Example 57, wherein the one or more anchors are coupled to the outlet end portion of the inner frame.

Example 59: The method of any example herein, in particular Example 57 or Example 58, wherein the outer frame is self-expanding.

Example 60: The method of any example herein, in particular Examples 51–59, wherein the native heart valve is a mitral heart valve or a tricuspid heart valve.

Example 61: A prosthetic heart valve for deployment to a native heart valve, the prosthetic heart valve comprising: a support structure having an inlet end portion and an outlet end portion and a passageway; a valve portion positioned within the passageway of the support structure, wherein the valve portion comprises a plurality of prosthetic valve leaflets, wherein the valve portion permits flow of blood through the passageway in one direction for replacing the function of the native heart valve; and/or one or more anchors extending radially outward from the support structure, the one or more anchors each having a hook shape and/or including a tip portion having a compliant braid.

Example 62: The prosthetic heart valve of any example herein, in particular Example 61, wherein the compliant braid comprises a wire braid.

Example 63: The prosthetic heart valve of any example herein, in particular Example 61 or Example 62, wherein each of the one or more anchors includes a strut arm, and/or the compliant braid extends from an axial end surface of the strut arm.

Example 64: The prosthetic heart valve of any example herein, in particular Examples 61–63, wherein each of the one or more anchors includes a strut arm, and/or the compliant braid covers a radially outward facing surface of the strut arm.

Example 65: The prosthetic heart valve of any example herein, in particular Example 64, wherein the compliant braid is offset to extend further from the strut arm in a radially outward direction than in a radially inward direction.

Example 66: The prosthetic heart valve of any example herein, in particular Examples 61–65, wherein the compliant braid has a bulb shape.

Example 67: The prosthetic heart valve of any example herein, in particular Examples 61–66, wherein the support structure includes an inner frame having an inlet end portion and an outlet end portion, and/or an outer frame including an inlet end portion that is coupled to the inlet end portion of the inner frame.

Example 68: The prosthetic heart valve of any example herein, in particular Example 67, wherein the one or more anchors are coupled to the outlet end portion of the inner frame.

Example 69: The prosthetic heart valve of any example herein, in particular Example 67 or Example 68, wherein the outer frame is self-expanding.

Example 70: The prosthetic heart valve of any example herein, in particular Examples 61–69, wherein the prosthetic heart valve is a replacement mitral heart valve or a replacement tricuspid heart valve.

Example 71: A method comprising: deploying a prosthetic heart valve to a native heart valve, the prosthetic heart valve including: a support structure having an inlet end portion and an outlet end portion and a passageway, a valve portion positioned within the passageway of the support structure, wherein the valve portion comprises a plurality of prosthetic valve leaflets, wherein the valve portion permits flow of blood through the passageway in one direction for replacing the function of the native heart valve, and/or one or more anchors extending radially outward from the support structure, the one or more anchors each having a hook shape and/or including a tip portion having a compliant braid.

Example 72: The method of any example herein, in particular Example 71, wherein the compliant braid comprises a wire braid.

Example 73: The method of any example herein, in particular Example 71 or Example 72, wherein each of the one or more anchors includes a strut arm, and/or the compliant braid extends from an axial end surface of the strut arm.

Example 74: The method of any example herein, in particular Examples 71–73, wherein each of the one or more anchors includes a strut arm, and/or the compliant braid covers a radially outward facing surface of the strut arm.

Example 75: The method of any example herein, in particular Example 74, wherein the compliant braid is offset to extend further from the strut arm in a radially outward direction than in a radially inward direction.

Example 76: The method of any example herein, in particular Examples 71–75, wherein the compliant braid has a bulb shape.

Example 77: The method of any example herein, in particular Examples 71–76, wherein the support structure includes an inner frame having an inlet end portion and an outlet end portion, and an outer frame including an inlet end portion that is coupled to the inlet end portion of the inner frame.

Example 78: The method of any example herein, in particular Example 77, wherein the one or more anchors are coupled to the outlet end portion of the inner frame.

Example 79: The method of any example herein, in particular Example 77 or Example 78, wherein the outer frame is self-expanding.

Example 80: The method of any example herein, in particular Examples 71–79, wherein the native heart valve is a mitral heart valve or a tricuspid heart valve.

Example 81: A prosthetic heart valve for deployment to a native heart valve, the prosthetic heart valve comprising: a support structure having an inlet end portion and an outlet end portion and a passageway; a valve portion positioned within the passageway of the support structure, wherein the valve portion comprises a plurality of prosthetic valve leaflets, wherein the valve portion permits flow of blood through the passageway in one direction for replacing the function of the native heart valve; and/or one or more anchors extending radially outward from the support structure, the one or more anchors each having a hook shape and/or including a tip portion having a loop.

Example 82: The prosthetic heart valve of any example herein, in particular Example 81, wherein the loop increases a flexibility of the tip portion.

Example 83: The prosthetic heart valve of any example herein, in particular Example 81 or Example 82, wherein each of the one or more anchors includes an anchor arm, and/or the loop comprises continuously extending material bounding an interior space.

Example 84: The prosthetic heart valve of any example herein, in particular Examples 81–83, wherein the loop includes a core, a core covering extending over the core, and/or a lubricious coating extending over the core covering.

Example 85: The prosthetic heart valve of any example herein, in particular Examples 81–84, further comprising a capture indicator at the tip portion of the loop, the capture indicator for indicating capture of a leaflet of the native heart valve by the respective one of the one or more anchors.

Example 86: The prosthetic heart valve of any example herein, in particular Examples 81–85, wherein the loop is deflectable in a radially inward direction.

Example 87: The prosthetic heart valve of any example herein, in particular Examples 81–86, wherein the support structure includes an inner frame having an inlet end portion and an outlet end portion, and/or an outer frame including an inlet end portion that is coupled to the inlet end portion of the inner frame.

Example 88: The prosthetic heart valve of any example herein, in particular Example 87, wherein the one or more anchors are coupled to the outlet end portion of the inner frame.

Example 89: The prosthetic heart valve of any example herein, in particular Example 87 or Example 88, wherein the outer frame is self-expanding.

Example 90: The prosthetic heart valve of any example herein, in particular Examples 81–89, wherein the prosthetic heart valve is a replacement mitral heart valve or a replacement tricuspid heart valve.

Example 91: A method comprising: deploying a prosthetic heart valve to a native heart valve, the prosthetic heart valve including: a support structure having an inlet end portion and an outlet end portion and a passageway, a valve portion positioned within the passageway of the support structure, wherein the valve portion comprises a plurality of prosthetic valve leaflets, wherein the valve portion permits flow of blood through the passageway in one direction for replacing the function of the native heart valve, and/or one or more anchors extending radially outward from the support structure, the one or more anchors each having a hook shape and/or including a tip portion having a loop.

Example 92: The method of any example herein, in particular Example 91, wherein the loop increases a flexibility of the tip portion.

Example 93: The method of any example herein, in particular Example 91 or Example 92, wherein each of the one or more anchors includes an anchor arm, and/or the loop comprises continuously extending material bounding an interior space.

Example 94: The method of any example herein, in particular Examples 91–93, wherein the loop includes a core, a core covering extending over the core, and/or a lubricious coating extending over the core covering.

Example 95: The method of any example herein, in particular Examples 91–94, further comprising a capture indicator at the tip portion of the loop, the capture indicator for indicating capture of a leaflet of the native heart valve by the respective one of the one or more anchors.

Example 96: The method of any example herein, in particular Examples 91–95, wherein the loop is deflectable in a radially inward direction.

Example 97: The method of any example herein, in particular Examples 91–96, wherein the support structure includes an inner frame having an inlet end portion and an outlet end portion, and/or an outer frame including an inlet end portion that is coupled to the inlet end portion of the inner frame.

Example 98: The method of any example herein, in particular Example 97, wherein the one or more anchors are coupled to the outlet end portion of the inner frame.

Example 99: The method of any example herein, in particular Example 97 or Example 98, wherein the outer frame is self-expanding.

Example 100: The method of any example herein, in particular Examples 91–99, wherein the native heart valve is a mitral heart valve or a tricuspid heart valve.

Example 101: A delivery system for a prosthetic heart valve, the delivery system comprising: a catheter shaft for advancement of the prosthetic heart valve to a treatment or implantation site and extending along an axis, the catheter shaft including a proximal end portion and a distal end portion, at least a portion of the catheter shaft including a retention area for the prosthetic heart valve; and/or an actuation mechanism for rotating the prosthetic heart valve about the axis of the catheter shaft.

Example 102: The delivery system of any example herein, in particular Example 101, wherein the actuation mechanism is adapted to rotate the prosthetic heart valve about the axis of the catheter shaft relative to at least a portion of the catheter shaft.

Example 103: The delivery system of any example herein, in particular Example 101 or Example 102, wherein the catheter shaft includes a capsule extending over the retention area, and/or the actuation mechanism is adapted to rotate the prosthetic heart valve relative to the capsule.

Example 104: The delivery system of any example herein, in particular Examples 101–103, wherein the actuation mechanism includes a motor for causing the prosthetic heart valve to rotate about the axis of the catheter shaft.

Example 105: The delivery system of any example herein, in particular Example 104, wherein the motor is positioned at the distal end portion of the catheter shaft.

Example 106: The delivery system of any example herein, in particular Examples 101–105, wherein the actuation mechanism is adapted to rotate a retainer for retaining the prosthetic heart valve to the catheter shaft.

Example 107: The delivery system of any example herein, in particular Example 106, wherein the actuation mechanism is adapted to rotate the retainer and/or the prosthetic heart valve with the prosthetic heart valve retained to the retainer.

Example 108: The delivery system of any example herein, in particular Examples 101–107, wherein the actuation mechanism is adapted to convert a longitudinal force along a length of the catheter shaft to a rotational force about the axis to thereby rotate the prosthetic heart valve about the axis of the catheter shaft.

Example 109: The delivery system of any example herein, in particular Examples 101–108, further comprising the prosthetic heart valve, wherein the prosthetic heart valve includes anchors each having a hook shape for capturing a leaflet of a native heart valve, and/or wherein rotation of the prosthetic heart valve by the actuation mechanism rotates the anchors relative to the leaflet of the native heart valve.

Example 110: The delivery system of any example herein, in particular Examples 101–109, wherein the delivery system comprises a mitral replacement heart valve delivery system or a tricuspid replacement heart valve delivery system.

Example 111: A method comprising: deploying a prosthetic heart valve to a native heart valve utilizing a delivery system, the delivery system including: a catheter shaft for advancement of the prosthetic heart valve to the native heart valve and extending along an axis, the catheter shaft including a proximal end portion and a distal end portion, at least a portion of the catheter shaft including a retention area for the prosthetic heart valve, and/or an actuation mechanism for rotating the prosthetic heart valve about the axis of the catheter shaft.

Example 112: The method of any example herein, in particular Example 111, wherein the actuation mechanism is adapted to rotate the prosthetic heart valve about the axis of the catheter shaft relative to at least a portion of the catheter shaft.

Example 113: The method of any example herein, in particular Example 111 or Example 112, wherein the catheter shaft includes a capsule extending over the retention area, and/or the actuation mechanism is adapted to rotate the prosthetic heart valve relative to the capsule.

Example 114: The method of any example herein, in particular Examples 111–113, wherein the actuation mechanism includes a motor for causing the prosthetic heart valve to rotate about the axis of the catheter shaft.

Example 115: The method of any example herein, in particular Example 114, wherein the motor is positioned at the distal end portion of the catheter shaft.

Example 116: The method of any example herein, in particular Examples 111–115, wherein the actuation mechanism is adapted to convert a longitudinal force along a length of the catheter shaft to a rotational force about the axis to thereby rotate the prosthetic heart valve about the axis of the catheter shaft.

Example 117: The method of any example herein, in particular Examples 111–116, wherein the actuation mechanism is adapted to rotate a retainer for retaining the prosthetic heart valve to the catheter shaft.

Example 118: The method of any example herein, in particular Example 117, wherein the actuation mechanism is adapted to rotate the retainer and/or the prosthetic heart valve with the prosthetic heart valve retained to the retainer.

Example 119: The method of any example herein, in particular Examples 111–118, wherein the prosthetic heart valve includes anchors each having a hook shape for capturing a leaflet of a native heart valve, and/or wherein rotation of the prosthetic heart valve by the actuation mechanism rotates the anchors relative to the leaflet of the native heart valve.

Example 120: The method of any example herein, in particular Examples 111–119, wherein the native heart valve is a mitral heart valve or a tricuspid heart valve.

Example 121: A delivery system for a prosthetic heart valve, the delivery system comprising: a catheter shaft for advancement of the prosthetic heart valve to a treatment or implantation site and extending along an axis, the catheter shaft including a proximal end portion and a distal end portion, at least a portion of the catheter shaft including a retention area for the prosthetic heart valve; and/or a motor positioned at the distal end portion of the catheter shaft and/or adapted to cause a variation in a depth of the prosthetic heart valve along the axis relative to at least a portion of the catheter shaft.

Example 122: The delivery system of any example herein, in particular Example 121, further comprising a transmission assembly for transmitting a force provided by the motor to vary the depth of the prosthetic heart valve along the axis.

Example 123: The delivery system of any example herein, in particular Example 122, wherein the transmission assembly includes one or more gears transmitting the force provided by the motor to vary the depth of the prosthetic heart valve along the axis.

Example 124: The delivery system of any example herein, in particular Example 123, wherein the one or more gears comprise one or more worm gears.

Example 125: The delivery system of any example herein, in particular Examples 121–124, wherein the catheter shaft includes a capsule for extending over the prosthetic heart valve, and/or the motor is adapted to vary the depth of the prosthetic heart valve relative to the capsule.

Example 126: The delivery system of any example herein, in particular Examples 121–125, further comprising a controller for operating the motor.

Example 127: The delivery system of any example herein, in particular Example 126, further comprising one or more control conduits extending along the catheter shaft and/or coupling the controller to the motor.

Example 128: The delivery system of any example herein, in particular Examples 121–127, wherein the motor is adapted to produce a rotational force that produces a linear motion of the prosthetic heart valve.

Example 129: The delivery system of any example herein, in particular Examples 121–128, further comprising the prosthetic heart valve, wherein the prosthetic heart valve includes anchors each having a hook shape for capturing a leaflet of a native heart valve, and/or wherein rotation of the prosthetic heart valve by the actuation mechanism rotates the anchors relative to the leaflet of the native heart valve.

Example 130: The delivery system of any example herein, in particular Examples 121–129, wherein the delivery system comprises a mitral replacement heart valve delivery system or a tricuspid replacement heart valve delivery system.

Example 131: A method comprising: deploying a prosthetic heart valve to a native heart valve utilizing a delivery system, the delivery system including: a catheter shaft for advancement of the prosthetic heart valve to the native heart valve and extending along an axis, the catheter shaft including a proximal end portion and a distal end portion, at least a portion of the catheter shaft including a retention area for the prosthetic heart valve, and/or a motor positioned at the distal end portion of the catheter shaft and/or adapted to cause a variation in a depth of the prosthetic heart valve along the axis relative to at least a portion of the catheter shaft.

Example 132: The method of any example herein, in particular Example 131, wherein a transmission assembly transmits a force provided by the motor to vary the depth of the prosthetic heart valve along the axis.

Example 133: The method of any example herein, in particular Example 132, wherein the transmission assembly includes one or more gears transmitting the force provided by the motor to vary the depth of the prosthetic heart valve along the axis.

Example 134: The method of any example herein, in particular Example 133, wherein the one or more gears comprise one or more worm gears.

Example 135: The method of any example herein, in particular Examples 131–134, wherein the catheter shaft includes a capsule for extending over the prosthetic heart valve, and/or the motor is adapted to vary the depth of the prosthetic heart valve relative to the capsule.

Example 136: The method of any example herein, in particular Examples 131–135, further comprising utilizing a controller to operate the motor.

Example 137: The method of any example herein, in particular Example 136, wherein one or more control conduits extend along the catheter shaft and/or coupling the controller to the motor.

Example 138: The method of any example herein, in particular Examples 131–137, wherein the motor is adapted to produce a rotational force that produces a linear motion of the prosthetic heart valve.

Example 139: The method of any example herein, in particular Examples 131–138, wherein the prosthetic heart valve includes anchors each having a hook shape for capturing a leaflet of a native heart valve, and/or wherein rotation of the prosthetic heart valve by the actuation mechanism rotates the anchors relative to the leaflet of the native heart valve.

Example 140: The method of any example herein, in particular Examples 131–139, wherein the native heart valve is a mitral heart valve or a tricuspid heart valve.

Example 141: A delivery system for a prosthetic heart valve, the delivery system comprising: a catheter shaft for advancement of the prosthetic heart valve to a treatment or implantation site, the catheter shaft including a proximal end portion and a distal end portion, the catheter shaft including a nose body and a sheath having a distal end portion attached to the nose body, the sheath including an interior lumen for a guide wire to pass through, and a portion of the catheter shaft including a retention area for the prosthetic heart valve; and/or an imaging system coupled to one or more of the nose body or the sheath, the imaging system adapted to image vasculature of a subject’s body from interior of the subject’s vasculature.

Example 142: The delivery system of any example herein, in particular Example 141, wherein the imaging system comprises an optical coherence tomograph imaging system.

Example 143: The delivery system of any example herein, in particular Example 141 or Example 142, wherein the imaging system comprises an ultrasound imaging system.

Example 144: The delivery system of any example herein, in particular Examples 141–143, wherein the nose body is adapted to translate axially relative to another portion of the catheter shaft.

Example 145: The delivery system of any example herein, in particular Examples 141–144, wherein the sheath is adapted to translate axially relative to another portion of the catheter shaft.

Example 146: The delivery system of any example herein, in particular Examples 141–145, further comprising one or more signal conduits extending along the catheter shaft and adapted to transfer signals from the imaging system.

Example 147: The delivery system of any example herein, in particular Examples 141–146, wherein the catheter shaft includes a capsule sheath for extending over the retention area.

Example 148: The delivery system of any example herein, in particular Examples 141–147, further comprising a handle positioned at the proximal end portion of the catheter shaft.

Example 149: The delivery system of any example herein, in particular Examples 141–148, further comprising the prosthetic heart valve, wherein the prosthetic heart valve includes anchors each having a hook shape for capturing a leaflet of a native heart valve.

Example 150: The delivery system of any example herein, in particular Examples 141–149, wherein the delivery system comprises a mitral replacement heart valve delivery system or a tricuspid replacement heart valve delivery system.

Example 151: A method comprising: imaging vasculature of a subject’s body from interior of the subject’s vasculature utilizing an imaging system, the imaging system being coupled to one or more of a nose body or a sheath of a catheter shaft, the catheter shaft being for advancement of a prosthetic heart valve to a treatment or implantation site, the catheter shaft including a proximal end portion and a distal end portion, the catheter shaft including the nose body and the sheath, the sheath having a distal end portion attached to the nose body and the sheath having an interior lumen for a guide wire to pass through, and/or a portion of the catheter shaft including a retention area for the prosthetic heart valve.

Example 152: The method of any example herein, in particular Example 151, wherein the imaging system comprises an optical coherence tomograph imaging system.

Example 153: The method of any example herein, in particular Example 151 or Example 152, wherein the imaging system comprises an ultrasound imaging system.

Example 154: The method of any example herein, in particular Examples 151–153, wherein the nose body is adapted to translate axially relative to another portion of the catheter shaft.

Example 155: The method of any example herein, in particular Examples 151–154, wherein the sheath is adapted to translate axially relative to another portion of the catheter shaft.

Example 156: The method of any example herein, in particular Examples 151–155, wherein one or more signal conduits extend along the catheter shaft and are for transferring signals from the imaging system.

Example 157: The method of any example herein, in particular Examples 151–156, wherein the catheter shaft includes a capsule sheath for extending over the retention area.

Example 158: The method of any example herein, in particular Examples 151–157, wherein a handle is positioned at the proximal end portion of the catheter shaft.

Example 159: The method of any example herein, in particular Examples 151–158, wherein the prosthetic heart valve includes anchors each having a hook shape for capturing a leaflet of a native heart valve.

Example 160: The method of any example herein, in particular Examples 151–159, wherein the catheter shaft is of a mitral replacement heart valve delivery system or a tricuspid replacement heart valve delivery system.

Example 161: A guide wire system for a prosthetic heart valve delivery system, the guide wire system comprising: a guide wire body having a proximal end portion and a distal end portion; and an imaging system coupled to the guide wire body, the imaging system adapted to image vasculature of a subject’s body from interior of the subject’s vasculature.

Example 162: The guide wire system of any example herein, in particular Example 161, wherein the imaging system comprises an optical coherence tomograph imaging system.

Example 163: The guide wire system of any example herein, in particular Example 161 or Example 162, wherein the imaging system comprises an ultrasound imaging system.

Example 164: The guide wire system of any example herein, in particular Examples 161–163, wherein the distal end portion of the guide wire body includes an atraumatic tip.

Example 165: The guide wire system of any example herein, in particular Examples 161–164, further comprising one or more conduits extending along the guide wire body and/or for transmitting signals from the imaging system to a processor.

Example 166: A method comprising: imaging vasculature of a subject’s body from interior of the subject’s vasculature utilizing an imaging system, the imaging system being coupled to a guide wire body.

Example 167: The method of any example herein, in particular Example 166, wherein the imaging system comprises an optical coherence tomograph imaging system.

Example 168: The method of any example herein, in particular Example 166 or Example 167, wherein the imaging system comprises an ultrasound imaging system.

Example 169: The method of any example herein, in particular Examples 166–168, wherein a distal end portion of the guide wire body includes an atraumatic tip.

Example 170: The method of any example herein, in particular Examples 166–169, wherein one or more conduits extend along the guide wire body for transmitting signals from the imaging system to a processor.

Example 171: A device for use at or deployment to a native heart valve, the device comprising (1) a support structure comprising a proximal end portion and a distal end portion; and (2) one or more anchors extending or capable of extending radially outward from the support structure, wherein the one or more anchors comprise one or more of the following: (a) hook shape, (b) an anchor arm that is forked to form a plurality of prongs diverging from each other circumferentially, (c) a tip portion with an undulation for increasing a flexibility of the tip portion, (d) a tip portion having a braid, and/or (e) a tip portion having a loop.

Example 172: The device of example 171, wherein the proximal end portions is an inlet end portion, the distal end portion is an outlet end portion, and/or a passageway extends between the inlet end portion and the outlet end portion.

Example 173: The device of any one of examples 171-172, wherein the support structure comprises an inner frame having a proximal end portion and a distal end portion.

Example 174: The device of example 173, wherein the support structure further comprises an outer frame including a proximal end portion.

Example 175: The device of example 174, wherein the proximal end portion of the outer frame is coupled to the proximal end portion of the inner frame

Example 176: The device of any one of claims 171-175, wherein the support structure comprises a plateau portion that extends radially outward from the proximal end portion of the support structure to a shoulder of the support structure.

Example 177: The device of example 176, wherein the support structure comprises an axially extending portion that extends axially from the shoulder to the distal end portion of the support structure, the shoulder angling the plateau portion relative to the axially extending portion.

Example 178: The device of any one of examples 171-177, wherein the plateau portion includes a flexibility feature that allows the plateau portion to deflect for axial translation of the inner frame relative to the shoulder.

Example 179: The device of example 178, wherein the flexibility feature comprises a portion of a strut of the support structure that is more flexible than an adjacent portion of the strut.

Example 180: The device of any one of examples 178-179, wherein the flexibility feature comprises an undulation of a strut of the outer frame.

Example 181: The device of any one of examples 171-180, wherein the device is a prosthetic heart valve deployable at a native heart valve.

Example 182: The device of any one of examples 171-181, further comprising a valve portion is positioned within a passageway of the support structure, wherein the valve portion permits flow of blood through the passageway in one direction but inhibits flow of blood in an opposite direction.

Example 183: The device of example 182, wherein the valve portion comprises a plurality of prosthetic valve leaflets.

Example 184: The device of any one of examples 171-183, further comprising one or more anchors coupled to the distal end portion of the support structure.

Example 185: The device of example 184, wherein the one or more anchors each have a hook shape.

Example 186: The device of any one of examples 184-185, wherein the one or more anchors each include an anchor arm that is forked to form a plurality of prongs diverging from each other.

Example 187: The device of example 186, wherein the anchor arm includes a split portion that forks the anchor arm, and/or a stem portion positioned radially inward of the split portion.

Example 188: The device of example 187, wherein the stem portion has a circumferential width that is the same as or greater than a combined circumferential width of each of the plurality of prongs.

Example 189: The device of any one of examples 187 or 188, wherein the one or more anchors include a plurality of the anchors, and/or the plurality of prongs of the plurality of the anchors extend over at least about 120 degrees of a circumference of the device.

Example 190: The device of any one of examples 184-189, wherein the one or more anchors each include a tip portion with an undulation for increasing a flexibility of the tip portion.

Example 191: The device of example 190, wherein the undulation extends in a circumferential direction on the tip portion.

Example 192: The device of any one of examples 190-191, wherein the one or more anchors each include a strut arm, the strut arm having the tip portion, the tip portion of the strut arm having a circumferential width that is greater than a radial thickness of the tip portion of the strut arm.

Example 193: The device of any one of examples 190-192, wherein the undulation increases a flexibility of the tip portion in a radially inward direction.

Example 194: The device of any one of examples 184-193, wherein the one or more anchors each include a tip portion having a braid.

Example 195: The device of example 194, wherein the braid is a compliant braid

Example 196: The device of example 195, wherein the compliant braid is a wire braid.

Example 197: The device of any one of examples 194-196, wherein the one or more anchors each include a strut arm, and/or the braid covers a radially outward facing surface of the strut arm.

Example 198: The device of example 197, wherein the braid is offset to extend further from the strut arm in a radially outward direction than in a radially inward direction.

Example 199: The device of any one of examples 184-198, wherein the one or more anchors each include a tip portion having a loop.

Example 200: The device of example 199, wherein the loop increases a flexibility of the tip portion.

Example 201: The device of any one of examples 199 or 200, wherein the one or more anchors each include an anchor arm, and/or the loop comprises continuously extending material bounding an interior space.

Example 202: The device of any one of examples 199–201, wherein the loop includes a core, a core covering extending over the core, and/or a lubricious coating extending over the core covering.

Example 203: A delivery system for a device, the delivery system comprising a catheter shaft for advancement of the device to a treatment site inside a subject (e.g., inside a living animal or inside a simulation) and extending along an axis, the catheter shaft including a proximal end portion and a distal end portion, at least a portion of the catheter shaft including a retention area for the device.

Example 204: The delivery system of example 203, further comprising an actuation mechanism for rotating the device about the axis of the catheter shaft.

Example 205: The delivery system of example 204, wherein the actuation mechanism is adapted to rotate the device about the axis of the catheter shaft relative to at least a portion of the catheter shaft.

Example 206: The delivery system of any one of examples 203-205, wherein the catheter shaft includes a capsule extending over the retention area, and/or the actuation mechanism is adapted to rotate the device relative to the capsule.

Example 207: The delivery system of any one of examples 203–206, wherein the actuation mechanism includes a motor for causing the device to rotate about the axis of the catheter shaft.

Example 208: The delivery system of any one of examples 203–207, further comprising a motor positioned at the distal end portion of the catheter shaft and adapted to cause rotating of the device and/or a variation in a depth of the device along the axis relative to at least a portion of the catheter shaft.

Example 209: The delivery system of any one of examples 203–208, further comprising an imaging system coupled directly or indirectly to the catheter shaft, the imaging system adapted to image inside of a subject’s body from inside of the subject’s body.

Example 210: The delivery system of any one of examples 203–209, further comprising a guide wire system, the guide wire system comprising: (a) a guide wire body having a proximal end portion and a distal end portion; and (b) an imaging system coupled to the guide wire body, the imaging system adapted to image inside of a subject’s body from inside of the subject’s body.

Any of the features of any of the examples, including but not limited to any of the first through 170 examples referred to above, is applicable to all other aspects and examples identified herein, including but not limited to any examples of any of the first through 170 examples referred to above. Moreover, any of the features of an example of the various examples, including but not limited to any examples of any of the first through 170 examples referred to above, is independently combinable, partly or wholly with other examples described herein in any way, e.g., one, two, or three or more examples may be combinable in whole or in part. Further, any of the features of the various examples, including but not limited to any examples of any of the first through 170 examples referred to above, may be made optional to other examples. Any example of a method can be performed by a system or apparatus of another example, and any aspect or example of a system or apparatus can be configured to perform a method of another aspect or example, including but not limited to any examples of any of the first through 170 examples referred to above.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only and is not intended to limit the scope of systems, apparatuses, and methods as disclosed herein, which is defined solely by the claims. Accordingly, the systems, apparatuses, and methods are not limited to that precisely as shown and described.

Some implementations of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

The techniques, methods, processes, operations, steps, etc. described or suggested herein (e.g., in the disclosure or examples above) or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any

other reason. All statements as to the date or representation as to the contents of these documents is based on the information available to the applicants and does not constitute any admission as to the correctness of the dates or contents of these documents.

What is claimed is:

1. A delivery system for an implant for use at a native valve of a heart of a subject, the delivery system comprising:
a guidewire configured to be advanced to the native valve; and
a catheter shaft for advancement of the implant to the native valve including:
a lumen, which is advanceable over and along the guidewire;
a retention area for holding the implant; and
an engager that is configured to reversibly engage the guidewire such that rotation of the guidewire induces a rotation in the retention area and thereby the implant.

2. The delivery system according to claim 1, wherein the retention area is disposed at a distal portion of the shaft.

3. The delivery system according to claim 1, wherein rotation of the guidewire is controlled via a motor.

4. The delivery system according to claim 1, wherein the engager is disposed distal to the retention area.

5. The delivery system according to claim 1, wherein the engager is disposed at the retention area.

6. The delivery system according to claim 1, wherein the guidewire is coaxial with the catheter shaft.

7. The delivery system according to claim 1, further comprising an extracorporeal unit coupled to (i) a proximal portion of the catheter shaft, and (ii) a proximal portion of the guidewire, and is configured to rotate the guidewire relative to the catheter shaft.

8. The delivery system according to claim 1, wherein the guidewire includes an engagement feature that is configured to be engaged by the engager to rotationally lock the guidewire to the retention area.

9. The delivery system according to claim 8, wherein:
the engagement feature is a keyed portion of the guidewire; and
immediately distal and proximal to the engagement feature, the guidewire is smooth.

10. The delivery system according to claim 8, wherein the engagement feature includes a recessed area.

11. The delivery system according to claim 8, wherein the engager is biased toward the guidewire.

12. The delivery system according to claim 11, wherein the engager includes a compression spring.

13. The delivery system according to claim 11, wherein the biasing of the engager is such that the engager automatically engages the engagement feature upon longitudinal alignment of the engager with the engagement feature.

14. The delivery system according to claim 1, wherein the engager is a grip body.

15. The delivery system according to claim 14, wherein the grip body grips the guidewire to engage the guidewire.

16. The delivery system according to claim 15, wherein the grip body includes spikes configured to capture and rotationally lock onto the guidewire.

17. The delivery system according to claim 15, further comprising:
an actuator coupled to the grip body and configured to advance and withdraw the grip body; and
a curved retainer disposed on the catheter shaft that is configured to accept the grip body.

18. The delivery system according to claim 17, wherein the grip body engages the guidewire when the grip body is advanced, via the actuator, into the curved retainer.

19. The delivery system according to claim 17, wherein the actuator is coaxial with the catheter shaft.

20. The delivery system according to claim 17, wherein the grip body is disposed within the catheter shaft such that rotating the grip body rotates the catheter shaft.
